(19) United States
(12) Patent Application Publication
(10) Pub. No.: US 2017/0181703 A1
(43) Pub. Date: Jun. 29, 2017

(54) Monitoring A Garment

(71) Applicant: ZOLL Medical Corporation, Chelmsford, MA (US)

(72) Inventors: Thomas E. Kaib, Irwin, PA (US); Shane S. Volpe, Saltsburg, PA (US); Mark Jerome Owens, Wexford, PA (US); John Clark, Pittsburgh, PA (US)

(21) Appl. No.: 15/389,781

(22) Filed: Dec. 23, 2016

Related U.S. Application Data
(60) Provisional application No. 62/272,245, filed on Dec. 29, 2015.

(51) Int. Cl. A61B 5/00; A41D 1/00; A61N 1/362; A61N 1/04; A61B 5/0452; A61N 1/39; A41D 13/12; A61B 5/0408

(52) U.S. Cl. CPC A61B 5/6805; A41D 13/1245; A41D 13/1281; A41D 1/002; A61B 5/0022; A61B 5/0408; A61N 1/0484; A61B 5/0452; A61N 1/3987; A61N 1/3625

(57) ABSTRACT

An apparatus comprising: a first garment configured to be worn about a torso of a patient; and a first garment identification component disposed as a part of the first garment, the first garment identification component configured to operably couple with a medical device controller, wherein the medical device controller is configured to identify the first garment based on one or more values provided by the first garment identification component.

### CLAIM OF PRIORITY

This application claims priority under 35 USC §119(e) to U.S. Patent Application Ser. No. 62/272,245, filed on Dec. 29, 2015, the entire contents of which are hereby incorporated by reference.

### BACKGROUND

This disclosure relates to systems and techniques for monitoring a garment.

There are a wide variety of electronic and mechanical devices for monitoring and treating patients' medical conditions. In some examples, depending on the underlying medical condition being monitored or treated, medical devices such as cardiac pacemakers or defibrillators may be surgically implanted or connected externally to the patient. In some cases, physicians may use medical devices alone or in combination with drug therapies to treat patient medical conditions.

One of the most deadly cardiac arrhythmias is ventricular fibrillation, which occurs when normal, regular electrical impulses are replaced by irregular and rapid impulses, causing the heart muscle to stop normal contractions and to begin to quiver. Normal blood flow ceases, and organ damage or death can result in minutes if normal heart contractions are not restored. Because the victim has no perceptible warning of the impending fibrillation, death often occurs before the necessary medical assistance can arrive. Other cardiac arrhythmias can include excessively slow heart rates known as bradycardia.

Implantable or external pacemakers and defibrillators (such as automated external defibrillators or AEDs) have significantly improved the ability to treat these otherwise life-threatening conditions. Such devices operate by applying corrective electrical pulses directly to the patient's heart. For example, bradycardia can be corrected through the use of an implanted or external pacemaker device. Ventricular fibrillation can be treated by an implanted or external defibrillator.

For example, certain medical devices operate by continuously or substantially continuously monitoring the patient's heart through one or more sensing electrodes for treatable arrhythmias and, when such is detected, the device applies corrective electrical pulses to the heart through one or more therapy electrodes. For example, such a medical device is a wearable medical device, which includes a garment that is worn by the patient.

### SUMMARY

In one aspect, an apparatus includes a first garment configured to be worn about a torso of a patient. The apparatus also includes a first garment identification component disposed as a part of the first garment. The first garment identification component is configured to operably couple with a medical device controller. The medical device controller is configured to identify the first garment based on one or more values provided by the first garment identification component.

Implementations can include one or more of the following features.

In some implementations, the medical device controller is configured to associate with the first garment after identifying the first garment.

In some implementations, the apparatus includes a wearable medical device.

In some implementations, the first garment identification component includes at least one of a near field communication (NFC) element and a machine-readable data element.

In some implementations, the NFC element includes a radio-frequency identification (RFID) element.

In some implementations, the machine-readable data element includes a barcode.

In some implementations, the apparatus includes the medical device controller. The medical device controller is configured to determine an amount of time that the first garment has been worn by the patient.

In some implementations, the medical device controller is configured to determine an amount of time that the first garment has been worn by the patient by determining an amount of time that the first garment has been associated with the medical device controller without the medical device controller being associated with another garment.

In some implementations, the medical device controller is configured to determine that the first garment is to be removed or replaced based on the amount of time that the first garment has been associated with the medical device controller without the medical device controller being associated with another garment.

In some implementations, the medical device controller is configured to determine that the first garment is to be removed or replaced if the amount of time meets a predetermined threshold amount of time.

In some implementations, the medical device controller is configured to operably couple with a second garment identification component disposed as a part of a second garment configured to be worn about the torso of the patient. The medical device controller is configured to identify the second garment based on one or more values provided by the second garment identification component.

In some implementations, the medical device controller is configured to disassociate the medical device controller from the first garment and associate the medical device controller with the second garment upon the medical device controller identifying the second garment.

In some implementations, the medical device controller is configured to, upon identifying the second garment, provide a prompt to a user to enable the user to cause the medical device controller to disassociate the medical device controller from the first garment and associate the medical device controller with the second garment.

In another aspect, a wearable medical device includes a controller, a memory storing a first value identifying a first garment configured to couple with the controller, a communication interface, and one or more processors. The one or more processors are configured for receiving, from the communication interface, a second value identifying a second garment configured to couple with the controller. The one or more processors are also configured for comparing the first value and the second value. The one or more processors are also configured for, based on the comparison of the first value and the second value, storing data indicative of whether the first garment has been removed on at least a predetermined interval.

Implementations can include one or more of the following features.

In some implementations, one or both of the first value identifying the first garment and the second value identifying the second garment includes data that corresponds to an identity of the respective garment.

In some implementations, one or both of the first value identifying the first garment and the second value identifying the second garment is unique to the respective garment.

In some implementations, one or both of the first value identifying the first garment and the second value identifying the second garment includes data that corresponds to one or more of a serial number of the respective garment, a model number of the respective garment, and a size of the respective garment.

In some implementations, the data indicative of whether the first garment has been removed on at least a predetermined interval includes data indicative of whether the first garment has been replaced with the second garment on at least a predetermined interval.

In some implementations, the wearable medical device includes a user interface configured to provide one or more messages based at least in part on the data indicative of whether the first garment has been removed on at least a predetermined interval.

In some implementations, the one or more processors are configured for calculating the data indicative of whether the first garment has been removed on at least a predetermined interval. The calculating is based at least on the second value.

In some implementations, the wearable medical device includes a network communication interface configured to transmit, to a server in communication with a network, the data indicative of whether the first garment has been removed on at least a predetermined interval.

In another aspect, an apparatus includes a garment configured to be worn about a torso of a patient, a garment monitoring component disposed as a part of the garment and configured to determine whether the garment has been laundered according to a predetermined schedule, and a communication device operably connected to the garment monitoring component and configured to exchange information related to the laundering of the garment with a medical device controller.

Implementations can include one or more of the following features.

In some implementations, the garment monitoring component includes at least one sensor that measures at least one of temperature, humidity, pH, and fabric stretch.

In some implementations, the garment monitoring component is configured to determine whether the garment has been laundered according to a predetermined schedule based at least in part on two or more measured values.

In some implementations, the two or more measured values are temperature values measured by a temperature sensor, and the determination of whether the garment has been laundered according to the predetermined schedule is based on whether any of the temperature values meets a predetermined threshold.

In some implementations, a temperature value that meets the predetermined threshold is indicative of the garment having been in a laundering environment.

In some implementations, the two or more measured values are humidity values measured by a humidity sensor, and the determination of whether the garment has been laundered according to the predetermined schedule is based on whether any of the humidity values meets a predetermined threshold.

In some implementations, a humidity value that meets the predetermined threshold is indicative of the garment having been in a laundering environment.

In some implementations, the two or more measured values are pH values measured by a pH sensor, and the determination of whether the garment has been laundered according to the predetermined schedule is based on whether any of the pH values meets a predetermined threshold.

In some implementations, a pH value that meets the predetermined threshold is indicative of the garment having been in a laundering environment.

In some implementations, the two or more measured values are stretch values measured by a strain sensor, and the determination of whether the garment has been laundered according to the predetermined schedule is based on whether any of the stretch values meets a predetermined threshold.

In some implementations, a stretch value that meets the predetermined threshold is indicative of the garment having not been in a laundering environment.

In some implementations, a difference between the first stretch value and the second stretch value that meets the predetermined threshold is indicative of the garment having been in a laundering environment.

In some implementations, the strain sensor includes a coil embedded in fabric of the garment

In some implementations, the two or more measured values include one or both of an inductance value and a capacitance value indicative of stretch experienced by the garment.

In some implementations, the apparatus includes the medical device controller. The medical device controller includes a user interface configured to provide one or more messages based at least in part on the information related to the laundering of the garment.

In some implementations, the communication device is configured to transmit, to a server in communication with a network, the information related to the laundering of the garment.

In some implementations, the information related to the laundering of the garment includes one or more of a measured temperature value, humidity value, pH value, and stretch value.

In some implementations, one or more laundering events experienced by the garment are identified based on the information related to the laundering of the garment.

In some implementations, the communication device operates according to an NFC protocol.

In another aspect, an apparatus includes a garment configured to be worn about a torso of a patient, a garment monitoring component disposed as a part of the garment and configured to monitor a state of the garment, and a communication device operably connected to the garment monitoring component and configured to exchange information related to the state of the garment with a medical device controller.

Implementations can include one or more of the following features.

In some implementations, the apparatus includes a wearable medical device.

In some implementations, monitoring the state of the garment includes determining whether the garment is being cared for according to predetermined criteria.

In some implementations, the apparatus includes at least one sensing electrode for sensing a physiological condition of the patient.

In some implementations, the apparatus includes at least one cardiac sensing electrode for sensing a cardiac condition of the patient.

In some implementations, the apparatus includes at least one therapy electrode for delivering a therapy to the patient.

In some implementations, the apparatus includes at least one sensing electrode for sensing a cardiac arrhythmia condition of the patient, and at least one therapy electrode for delivering at least one therapeutic pulse to the patient in response to the sensed cardiac arrhythmia condition.

In some implementations, the apparatus includes a coil embedded in fabric of the garment. The coil is configured to provide to the garment monitoring component information related to a magnitude of stretch experienced by the garment.

In some implementations, the garment monitoring component is configured to monitor the state of the garment based at least in part on the information related to the magnitude of stretch.

In some implementations, the apparatus includes a tear sensor embedded in fabric of the garment. The tear sensor is configured to provide to the garment monitoring component information related to a tear in the garment.

In some implementations, the garment monitoring component is configured to monitor the state of the garment based at least in part on the information related to a tear in the garment.

In some implementations, the tear sensor includes a conductive mesh.

In some implementations, the information related to a tear in the garment is based at least in part on a magnitude of current running through the conductive mesh when a predetermined voltage is applied across the conductive mesh.

In some implementations, the garment monitoring component is configured to determine whether the garment is in an unsatisfactory condition based at least in part on one or more measured values related to a magnitude of stretch experienced by the garment, a magnitude of current running through a conductive mesh of the garment, or both.

In some implementations, the apparatus includes a slotted window through which a portion of the garment is visible. The visible portion of the garment is indicative of a magnitude of stretch experienced by the garment.

In some implementations, the apparatus includes an optical sensor configured to capture an image of the portion of the garment visible through the slotted window, and provide to the garment monitoring component information related to the image.

In some implementations, the information related to the image includes one or more values that represent a color composition of the image.

In some implementations, the apparatus includes the medical device controller. The medical device controller includes a user interface configured to provide one or more messages based at least in part on the state of the garment.

In some implementations, the communication device is configured to transmit, to a server in communication with a network, information related to the state of the garment.

In some implementations, the communication device operates according to an NFC protocol.

Implementations can include one or more of the following advantages.

In some implementations, the wearable medical device can determine an amount of time that a first garment has been associated with the medical device controller and infer how long the first garment has been worn by the patient and/or whether the first garment has been removed on at least a predetermined interval. Using such information, the wearable medical device can determine that the first garment should be removed and/or replaced with a second garment. The first garment can be replaced by the second garment either permanently (e.g., if the first garment is permanently damaged) or temporarily (e.g., if the first garment is temporarily in unsatisfactory condition due to excessive wear). In some implementations, the patient can wear the second garment while the first garment is laundered (e.g., to restore sufficient stretch/elasticity to the first garment).

In some implementations, the garment monitoring component is configured to determine whether the garment has been laundered according to a predetermined schedule and/or monitor the state of the garment. The state of the garment may be monitored based on information received from a strain sensor (e.g., a coil) indicative of a magnitude of stretch experienced by the garment. When the magnitude of stretch experienced by the garment meets or surpasses a predetermined threshold, it may be determined that the garment is in an unsatisfactory condition, and in particular, that the garment does not have sufficient elasticity for firmly pressing the electrodes against the skin of the patient.

In some implementations, the user interface can provide a message to the patient when it is determined that the garment is in an unsatisfactory condition. For example, if the garment monitoring component determines that the garment requires laundering and/or replacement, the medical device controller can present a message to the patient indicating such.

Other features and advantages of the examples described herein will be apparent from the drawings, detailed description, and claims.

### DESCRIPTION OF DRAWINGS

The accompanying drawings are not intended to be drawn to scale. In the drawings, components that are identical or nearly identical may be represented by a like numeral. For purposes of clarity, not ever component is labeled in every drawing. In the drawings:

FIG. 1 is an example of a wearable medical device that includes a medical device controller, a garment, and a garment monitoring component.

FIGS. 2A-2B show an example of the medical device controller of FIG. 1.

FIG. 3 is a functional schematic of the medical device controller of FIGS. 1 and 2A-2B.

FIG. 4 shows the medical device controller of FIGS. 1 and 2A-2B and an example of a base station.

FIG. 5 shows an example of the wearable medical device of FIG. 1 that includes a strain sensor.

FIGS. 6A and 7A show an example of an inductive element of the strain sensor in an unstretched position and a stretched position, respectively.

FIGS. 6B and 7B show an example of a capacitive element of the strain sensor in an unstretched position and a stretched position, respectively.

FIG. 8 shows an example of the wearable medical device of FIG. 1 that includes a tear sensor.

FIGS. 9A and 9B show an example of a conductive mesh of the tear sensor of FIG. 8.

FIG. 10 shows a functional schematic of the garment monitoring component of FIG. 1 providing information to an event log.

FIGS. 11A-11C show an example of a visual stretch detector that can be embedded in a garment.

FIGS. 12A-12B show another example of a visual stretch detector that can be embedded into a garment.

FIG. 13 shows an example of a pressure sensor that can be incorporated into an electrode of the wearable medical device.

### DETAILED DESCRIPTION

Described herein are systems and techniques for monitoring a garment of a medical device (e.g., a wearable medical device), such as a wearable monitoring and/or treatment device. For example, such a wearable monitoring device may be a wearable cardiac monitoring device or a wearable cardiac treatment device such as a wearable defibrillator. Sensing electrodes and/or treatment electrodes can be affixed to the garment. The garment can include an elastic material that, when worn by the patient, causes the electrodes to remain in firm contact against the patient's body. Intimate contact between the sensing electrodes and the patient's body facilitates the transfer of strong cardiac signals with minimal noise artifacts. In the case of treatment electrodes, intimate contact improves the efficiency of the treatment energy being delivered.

Over time, the condition of the garment may degrade for a variety of reasons. For example, among other reasons, the garment may lose its elasticity (either temporarily or permanently), become damaged, or become dirty. The diminished condition of the garment may have a negative impact on the efficacy of the accompanying electrodes. For example, if the garment experiences reduced elasticity due to excessive wear or develops a tear, the electrodes may not make sufficiently intimate contact with the patient's body, thereby resulting in diminished performance of the wearable defibrillator. Information related to the garment, including information related to the condition of the garment, can be provided by the wearable defibrillator so that the effectiveness of the wearable defibrillator can be monitored and/or maintained.

A medical device for use with the systems and techniques as disclosed herein can be configured to monitor one or more cardiac signals of a patient and determine whether the patient is experiencing a cardiac event. For example, the medical device can include a plurality of sensing electrodes that are disposed at various locations of the patient's body (e.g., the patient's upper body or torso) and configured to detect the cardiac signals of the patient. For example, such devices can be used as patient monitors, and more specifically, cardiac monitors in certain cardiac monitoring applications, such as Holter monitoring, mobile cardiac telemetry (MCT) and/or continuous event monitoring (CEM) applications.

In some implementations, the medical device can also be configured to determine an appropriate treatment for the patient based on the detected cardiac signals and cause one or more therapeutic pulses (e.g., defibrillating and/or pacing pulses) to be delivered to the heart of the patient. The medical device can include a plurality of therapy electrodes that are disposed at various locations of the patient's body and configured to deliver the therapeutic pulses. In some examples, the therapy electrodes can be integrated along with the sensing electrodes on a same electrode patch as described herein.

A medical device as described herein can use one or more electrodes for monitoring a patient for a cardiac arrhythmia condition such as bradycardia, ventricular tachycardia (VT) or ventricular fibrillation (VF). In addition, while the detection methods and systems described hereinafter are disclosed as detecting VT and VF, this is not to be construed as limiting the invention as other arrhythmias, such as, but not limited to, atrial arrhythmias such as premature atrial contractions (PACs), multifocal atrial tachycardia, atrial flutter, and atrial fibrillation, supraventricular tachycardia (SVT), junctional arrhythmias, tachycardia, junctional rhythm, junctional tachycardia, premature junctional contraction, and ventricular arrhythmias such as premature ventricular contractions (PVCs) and accelerated idioventricular rhythm may also be detected. In the case of treatment devices, such as pacing and/or defibrillating devices, if an arrhythmia condition is detected, the device can automatically provide a pacing, defibrillation, and/or Transcutaneous electrical nerve stimulation (TENS) pulses or shocks, as needed, to treat the condition.

Example Medical Devices

In some implementations, the medical device is an external medical device (e.g., in contrast to internal or implanted devices, such as implantable medical devices). For example, the external medical device can be configured as a wearable defibrillator, such as the LifeVest® wearable cardioverter defibrillator available from ZOLL® Medical Corporation of Chelmsford, Mass. The devices as described herein may be capable of continuous, substantially continuous, long-term and/or extended use or wear by, or attachment or connection to a patient. For example, devices as described herein may be capable of being used or worn by, or attached or connected to a patient, without substantial interruption for a predetermined period of time. In some examples, such devices may be capable of being used or worn by, or attached or connected to a patient for example, up to hours or beyond (e.g., weeks, months, or years). For example, devices as described herein may be capable of providing cardiac monitoring without substantial interruption for a predetermined period of time. In some examples, such devices may be capable of continuously or substantially continuously monitoring a patient for cardiac-related information (e.g., ECG information, arrhythmia information, heart sounds, etc.) and/or non-cardiac information (e.g., blood oxygen, the patient's temperature, glucose levels, and/or lung sounds), for example, up to hours or beyond (e.g., weeks, months, or years).

In some implementations, the medical device as described herein can be configured to monitor a patient presenting with syncope (e.g., by analyzing the patient's cardiac activity for aberrant patterns that can indicate abnormal physiological function). In some examples, aberrant patterns may occur prior to, during, or after the onset of syncope symptoms.

Example Wearable Medical Device

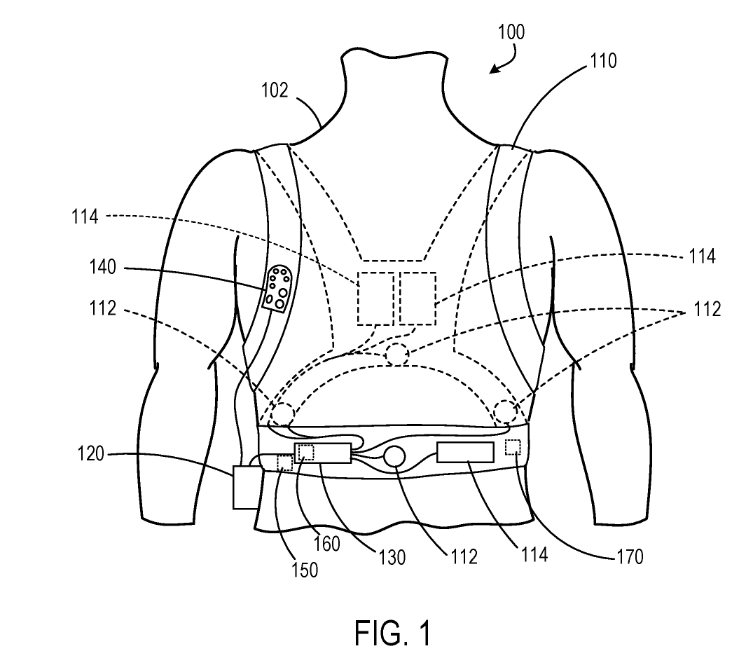

FIG. 1 illustrates an example medical device 100 that is wearable by a patient 102. The wearable medical device 100 includes a plurality of sensing electrodes 112 that can be disposed at various positions about the patient's body. The sensing electrodes 112 are electrically coupled to a medical device controller 120 through a connection pod 130. In some implementations, some of the components of the wearable medical device 100 are affixed to a garment 110 that can be worn on the patient's torso. For example, as shown in FIG. 1, the controller 120 can be mounted on a belt worn by the patient. The sensing electrodes 112 and connection pod 130 can be assembled or integrated into the garment 110 as shown. The sensing electrodes 112 are configured to monitor the cardiac function of the patient (e.g., by monitoring one or more cardiac signals of the patient). While FIG. 1 shows three sensing electrodes 112, fewer or additional sensing electrodes 112 may be provided, and the plurality of sensing electrodes 112 may be disposed at various locations about the patient's body.

The wearable medical device 100 can optionally include a plurality of therapy electrodes 114 that are electrically coupled (coupling not shown in the figure) to the medical device controller 120 through the connection pod 130. The therapy electrodes 114 are configured to deliver one or more therapeutic defibrillating shocks to the body of the patient if it is determined that such treatment is warranted. The connection pod 130 may include electronic circuitry and one or more sensors (e.g., a motion sensor, an accelerometer, etc.) that are configured to monitor patient activity. In some implementations, the wearable medical device 100 may be a monitoring only device that omits the therapy delivery capabilities and associated components (e.g., the therapy electrodes 114). In some implementations, various treatment components may be packaged into various components that can be attached or removed from the wearable medical device 100 as needed.

In some implementations, the wearable medical device 100 includes a communication interface, such as a wireless communication interface, for allowing components of the wearable medical device 100 to exchange information with each other. For example, the wearable medical device 100 may include Near Field Communication (NFC) elements. For example, such NFC elements can include short-range wireless technology protocols that enables devices to establish radio communication amongst each other in order to quickly exchange data over a low latency link (e.g., a link which has relatively low delay between transmission and receipt of a portion of data such as a data packet or frame). Some implementations of NFC techniques are based on standards defined by the International Electrotechnical Commission (IEC) and/or the International Organization for Standardization (ISO), for example, standards such as ISO 13157 and ISO 18092.

In some implementations, the NFC elements may be radio-frequency identification (RFID) elements. The garment 110 can include a garment identification component that includes one or more of such NFC elements that can store information related to an identity of the garment 110. In some implementations, an RFID reader 160 is disposed in the connection pod 130. An RFID tag 150 of the garment identification component can provide identity information to an RFID reader 160 when the RFID tag 150 is in proximity to the RFID reader 160. The RFID tag 150 can be powered by electromagnetic induction from magnetic fields produced by the RFID reader 160. In some implementations, the RFID tag 150 is a passive tag that is powered by interrogating radio waves transmitted by the RFID reader 160. The RFID reader 160 is configured to send a signal to the RFID tag 150 and receive a response signal that includes identification information related to the garment. The identification information can include one or more values. The one or more values can include data that corresponds to a serial number of the garment 110, a model number of the garment 110, and a size of the garment 110 (e.g., small, medium, large, extra-large), among others. The RFID reader 160 is configured to provide the identification information to the controller 120, and the controller 120 can associate with the garment based on the identification information. In this way, the controller 120 is configured to identify the garment 110 based on the one or more values provided by the garment identification component (e.g., the RFID tag 150) and determine when particular garments are associated/disassociated with the controller 120. In some implementations, the controller 120 is configured to associate with the garment 110 after (e.g., upon) the controller 120 identifying the garment 110.

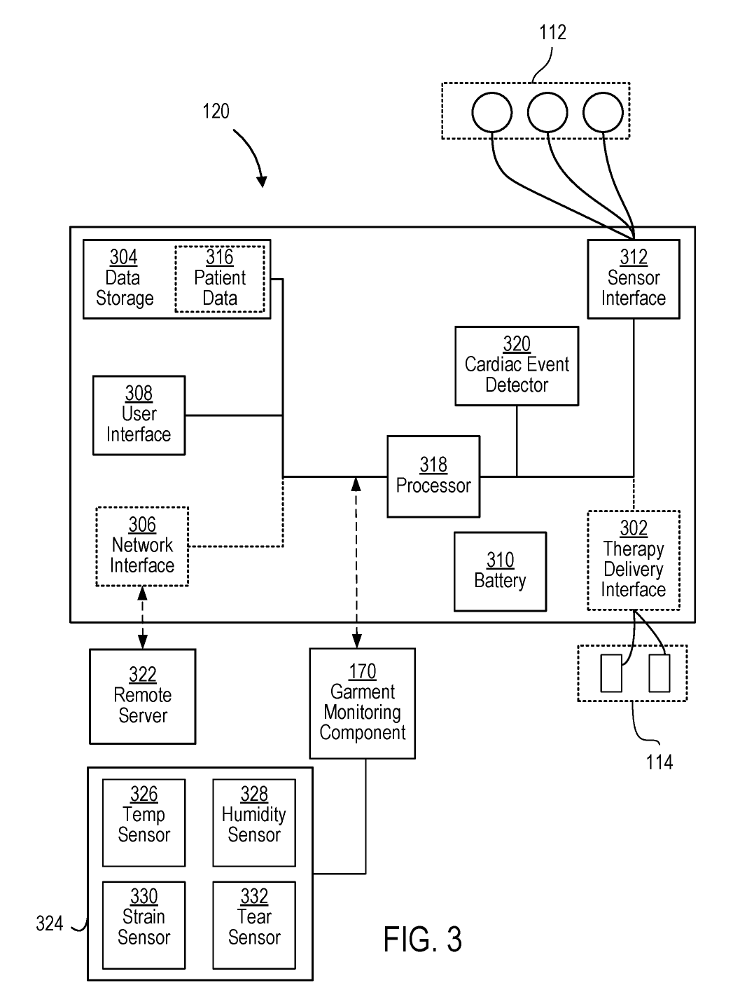

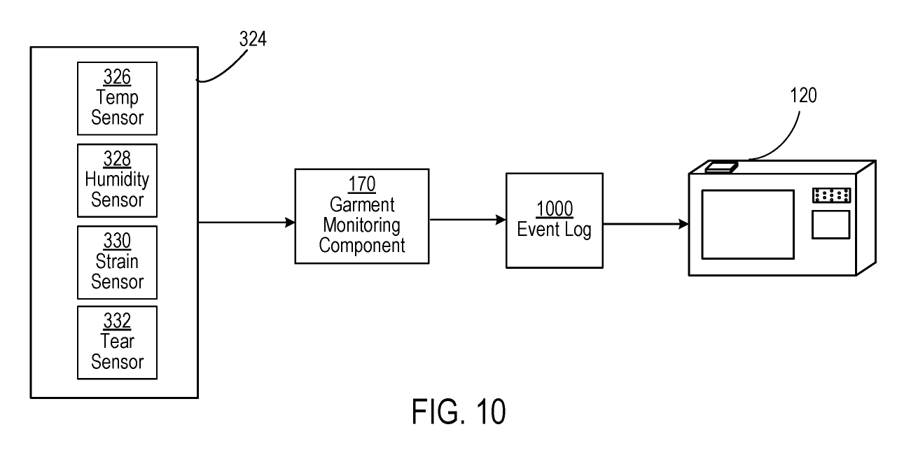

The wearable medical device 100 typically includes multiple garments that can be used with the wearable medical device 100. The patient can remove the garment 110 for laundering and replace it with a second garment. The second garment may also include an RFID tag for providing garment identification information to the RFID reader 160. The controller 120 can keep track of times and/or frequencies in which particular garments are associated with the controller 120 by storing information in a memory module (e.g., data storage 304 of FIG. 3). In some implementations, a log (e.g., the event log 1000 of FIG. 10) is maintained that includes identification information of all garments that have been associated with the controller 120, particular times at which each garment became associated with the controller 120, and/or particular times at which each garment became disassociated with the controller 120. In this way, the controller 120 can determine an amount of time that each garment has been associated with the controller 120. For example, if a first garment becomes associated with the controller 120 at t₀ and subsequently becomes disassociated with the controller 120 at t₁, the controller 120 determines that the first garment has been associated with the controller for an amount of time equal to t₁-t₀.

In some implementations, the controller 120 determines the amount of time that the first garment has been associated with the controller 120 by determining an amount of time that the first garment has been associated with the controller 120 without the controller 120 being associated with another garment. For example, if the first garment becomes associated with the controller 120 at t₀ and a second garment becomes associated with the controller 120 at t₁, the controller 120 determines that the first garment has been associated with the controller for an amount of time equal to t₁-t₀. The controller 120 can determine that the first garment is no longer associated with the controller 120 by comparing identification information of the garments. For example, the controller 120 may store a value (e.g., a serial number) identifying the first garment. When the second garment is in proximity to the RFID reader 160, the controller 120 can receive (e.g., from the corresponding RFID tag) a serial number identifying the second garment. The controller 120 can compare the stored value and the received value to determine that the first garment has been replaced with the second garment.

In some implementations, the controller 120 provides a prompt to the patient upon receiving identification information (e.g., from the RFID tag) related to a garment that is not currently associated with the controller 120. The prompt enables the patient to cause the controller 120 to disassociate from the first garment and associate with the second garment. The prompt may prevent garments from inadvertently being associated with the controller 120. For example, if a garment that is not intended to be associated with the controller 120 is inadvertently placed within wireless communication range of the RFID reader, the prompt may prohibit the garment from being paired with the controller 120 until an affirmative response to the prompt is received by the controller 120.

The controller 120 can infer that a particular garment has been worn for a particular amount of time based on the amount of time for which the particular garment has been associated with the controller 120. Thus, when a garment has been associated with the controller 120 for a predetermined threshold amount of time (e.g., one or two days) and/or when a garment has not been removed on at least a predetermined interval, the controller 120 may determine that the garment should be removed or replaced (e.g., with the second garment). For example, when a garment has been associated with the controller 120 for one or two days of continuous, uninterrupted use (e.g., use in which the garment has not been removed from the body), the controller 120 may determine that the garment should be removed or replaced (e.g., with the second garment). The controller 120 can store data indicative of whether the garment has been removed on at least the predetermined interval. The predetermined threshold amount of time and/or the predetermined interval may be based on historical information related to how long a garment can typically be worn before its condition is such that it should be replaced. As described in more detail below, the condition of the garment may call for it to be replaced due to a temporary loss of elasticity (e.g., such that the garment should be laundered) or a permanent loss of elasticity (e.g., such that the garment has reached the end of its useful life). For example, the predetermined threshold amount of time may be an amount of time after which garments typically temporarily lose their elasticities due to continuous wear. In such situations, the garment can be replaced with another garment. In some implementations, the garment can be laundered to regain sufficient elasticity. In some implementations, the predetermined threshold amount of time may be an amount of time after which garments typically permanently lose their elasticities. In such situations, the garment can be permanently replaced with a new garment.

In some implementations, the patient may alternate between wearing two garments. For example, the patient may wear a first garment for a particular (e.g., predetermined) amount of time (e.g., one or two days). After the particular amount of time, the garment may have temporarily developed an insufficient elasticity, and the patient may remove the first garment for laundering. While the first garment is being laundered (e.g., to restore its elasticity), a second garment may be worn by the patient. The second garment may be worn for a particular amount of time (e.g., one or two days). In this way, the first and second garments can be worn in an alternate fashion such that the patient is always in possession of a garment with sufficient elasticity.

In some implementations, the device may be configured with one or more default values for parameters relating to the predetermined amount of time. For example, the predetermined amount of time may be set to a default value of two days, e.g., the patient may be instructed to replace the garment every two days. A caregiver or other patient service representative may adjust these parameters based on the patient's unique circumstances.

In some implementations, the wearable medical device 100 is configured to transmit the garment identification information to a server in communication with a network. The controller 120 can include a network interface (306 of FIG. 3) that facilitates the communication of information between the controller 120 and one or more other devices or entities over a communications network. For example, the controller 120 can transmit identification information of all garments that have been associated with the controller 120, particular times at which each garment became associated with the controller 120, and/or particular times at which each garment became disassociated with the controller 120, among others. In some implementations, the controller 120 can transmit data indicative of whether a garment has been removed on at least a predetermined interval.

While the communication interface and the garment identification components have been described as being NFC and/or RFID elements, other elements can instead or additionally be used. For example, the garment can include a machine-readable data element such as a barcode, a matrix barcode, or a two-dimensional barcode, among others. The wearable medical device 100 can include a barcode reader that is configured to read the corresponding barcode. The barcode contains identification information related to the particular garment (e.g., the identification information described above).

In some implementations, the garment identification component may be an impedance-based element that is configured to interact with an impedance measuring circuit to provide identity information related to the garment 110. For example, the controller 120 may include an impedance measuring circuit and a programmable clock that may be interfaced to a processor (e.g., the processor 318 of FIG. 3) through, e.g., an analog-to-digital (A/D) converter. In operation, the impedance measuring circuit can receive a clock signal (e.g., in programmable periodic or aperiodic intervals), and apply the signal to the impedance-based garment identification component. For example, the garment identification component may be an electrical impedance element that has an impedance value that uniquely corresponds to the garment with which the garment identification component is associated, such as the garment 110 of FIG. 1. The magnitude of the response signal received from the garment identification component is monitored by the impedance measuring circuit. An impedance signal representative of the detected impedance is generated by the impedance measuring circuit. The generated signal may be a function of a ratio of the magnitudes of the applied and received signals (e.g., the attenuation of the signal). A look-up table of impedance values can be associated with unique identifiers corresponding to distinct garments. For example, an impedance of around 50 ohms may be associated in the table with a first garment while an impedance of around 75 ohms may be associated with a second garment. Such impedance values are exemplary values. The impedance signal representative of the impedance can be digitized by an A/D converter and provided to a processor for storage and/or further processing.

In some implementations, the impedance measuring circuit may be part of a garment monitoring component 170 that is operably coupled to the controller 120 via a wired or wireless connection. When the garment monitoring component 170 is operatively coupled to the controller 120, the garment monitoring component 170 and the controller 120 may be connected such that they are configured to communicate with either other based on the coupling method (e.g., via a wired or wireless connection). The garment monitoring component 170 may be disposed as part of the garment 110 or incorporated into the controller 120, as described in more detail below.

While the garment identification component has been described as an impedance-based element that is configured to interact with an impedance measuring circuit, in some implementations, the garment identification component may be a resistance-based element that is configured to interact with a resistance measuring circuit.

In some implementations, the communication interface is configured to use a serial protocol that uses a single data line and a ground reference to allow communication between a master device and a slave device. For example, such a protocol may be based on “1-Wire” technology. In some examples, a 1-Wire slave device may be incorporated into the garment 110 and operate as the garment identification component, and a 1-Wire master device may be disposed in the connection pod 130. In some implementations, the 1-Wire master device may be disposed elsewhere on the wearable medical device 100.

The communication employed between the master device and the slave device requires relatively little power to operate. In some examples, the master device includes a battery for powering the master device and providing parasitic power to the slave device via the data line. In some implementations, the slave device includes a capacitor for providing power to the slave device during times when the slave device is not in electrical contact with the master device. In this way, the master device and the slave device can operate for extended lengths of time (e.g., years) without requiring battery recharge or replacement.

The slave device includes an identification number (e.g., a 64-bit ID) that may be factory-programmed. When the data and ground terminals of the slave device are connected to the data and ground terminals of the master device, the master device provides power to the slave device, thereby initiating data communication (e.g., serial data communication) between the devices. In some examples, the leads of the master device have the form of conductive rings and the leads of the slave device have the form of conductive members, such that the slave device snaps into the master device (e.g., like a snap button). In this way, the master device and the slave device are in electrical communication when the slave device is snapped into the master device.

The identification number of the slave device can correspond to identification information related to the garment, such as the identification information described above. For example, the identification number can include one or more values that correspond to a serial number of the garment, a model number of the garment, and/or a size of the garment, among others. The master device can receive the identification number from the slave device when electrical communication is established and determine such identification information. In some implementations, the identification information is stored on the controller 120 (e.g., in the data storage 304 of FIG. 3). Upon receipt of the identification number of the slave device, the master device can access the identification information to determine the particular identification information that corresponds to the received identification number. When the slave device is removed from electrical communication with the master device, the slave device may enter and remain in a defined reset state until electrical communication is reestablished.

In some implementations, the wearable medical device 100 includes a garment monitoring component 170 that is disposed as part of the garment 110. The garment monitoring component 170 may be permanently disposed or removably disposed in the garment 110. For example, the garment monitoring component 170 may be stitched into fabric of the garment 110, disposed between layers (e.g., layers of fabric) of the garment 110, disposed in a compartment of the garment 110 (e.g., a pocket sewn into the garment 110), etc. In some implementations, the garment monitoring component 170 can be removed from the garment 110 prior to laundering (e.g., to prevent damage to the garment monitoring component 170). In some implementations (e.g., implementations in which the garment monitoring component 170 is permanently disposed into the garment 110), the garment monitoring component 170 may be waterproof and/or incorporated into a waterproof compartment of the garment 110.

The garment monitoring component 170 is operably coupled to the controller 120 via a wired or wireless connection. When the garment monitoring component 170 is operatively coupled to the controller 120, the garment monitoring component 170 and the controller 120 may be connected such that they are configured to communicate with either other based on the coupling method (e.g., via a wired or wireless connection). In some implementations, the garment monitoring component 170 includes a communication device that allows the garment monitoring component 170 to exchange information with the controller 120. The garment monitoring component 170 can exchange information with the controller 120 using a wireless technology protocol such as Bluetooth, NFC, RFID, or WiFi, to name a few. In some implementations, the garment monitoring component 170 is configured to removably connect to the connection pod 130 via a wired connection. The garment monitoring component 170 is configured to interact with one or more garment monitoring sensors (324 of FIG. 3). The garment monitoring component 170 is configured to receive information from the garment monitoring sensors 324 to determine a condition (e.g., an environmental condition, such as being in a laundering environment) and/or a state of the garment 110, as described in more detail below.

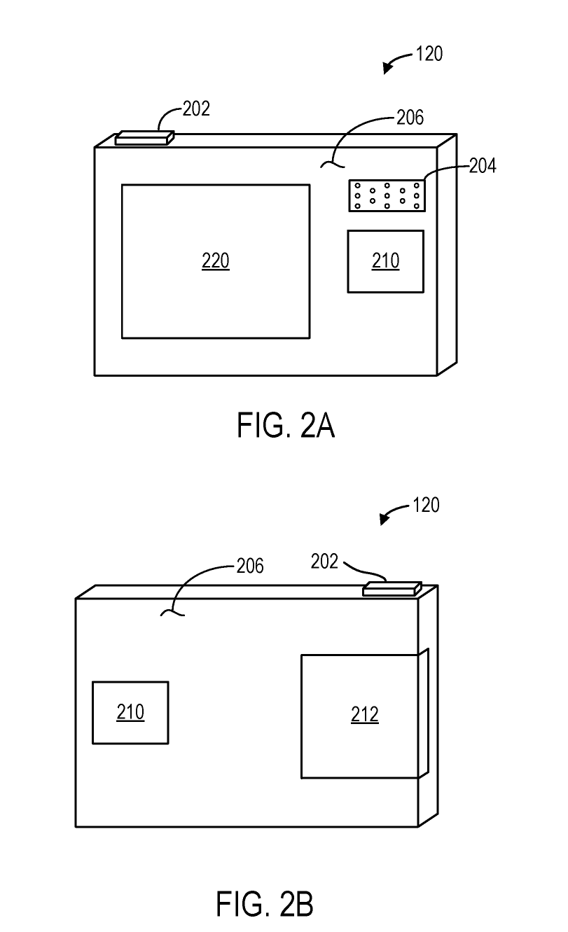

FIGS. 2A-2B illustrate an example of the medical device controller 120. The controller 120 may be powered by a rechargeable battery 212. The rechargeable battery 212 may be removable from a housing 206 of the medical device controller 120 to enable a patient and/or caregiver to swap a depleted (or near depleted) battery 212 for a charged battery. The controller 120 includes a user interface such as a touch screen 220 that can provide information to the patient, caregiver, and/or bystanders. The patient and/or caregiver can interact with the touch screen 220 to control the medical device 100. The controller 120 also includes a speaker 204 for communicating information to the patient, caregiver, and/or the bystander. The controller 120 includes one or more response buttons 210. In some examples, when the controller 120 determines that the patient is experiencing cardiac arrhythmia, the speaker 204 can issue an audible alarm to alert the patient and bystanders to the patient's medical condition. In some examples, the controller 120 can instruct the patient to press and hold one or both of the response buttons 210 to indicate that the patient is conscious, thereby instructing the medical device controller 120 to withhold the delivery of therapeutic defibrillating shocks. If the patient does not respond to an instruction from the controller 120, the medical device 100 may determine that the patient is unconscious and proceed with the treatment sequence, culminating in the delivery of one or more defibrillating shocks to the body of the patient. The medical device controller 120 may further include a port 202 to removably connect sensing devices (e.g., the sensing electrodes 112) and/or therapeutic devices (e.g., the therapy electrodes 114) to the medical device controller 120 (e.g., via the connection pod 130).

FIG. 3 shows a schematic of an example of the medical device controller 120 of FIGS. 1, 2A, and 2B. The controller 120 includes at least one processor 318, a sensor interface 312, an optional therapy delivery interface 302, data storage 304 (which may include patient data storage 316), an optional network interface 306, a user interface 308 (e.g., including the touch screen 220 shown in FIG. 2), and a battery 310. The sensor interface 312 may be coupled to any one or combination of sensors to receive information indicative of patient parameters. For example, the sensor interface 312 may be coupled to one or more sensing devices including, for example, the sensing electrodes 112. The therapy delivery interface 302 (if included) may be coupled to one or more electrodes that provide therapy to the patient including, for example, the therapy electrodes 114. In some implementations, the therapy delivery interface 302 can also be coupled to pacing electrodes and/or transcutaneous electrical nerve stimulation (TENS) electrodes. The sensor interface 312 and the therapy delivery interface 302 may implement a variety of coupling and communication techniques for facilitating the exchange of data between the sensors and/or therapy delivery devices and the controller 120.

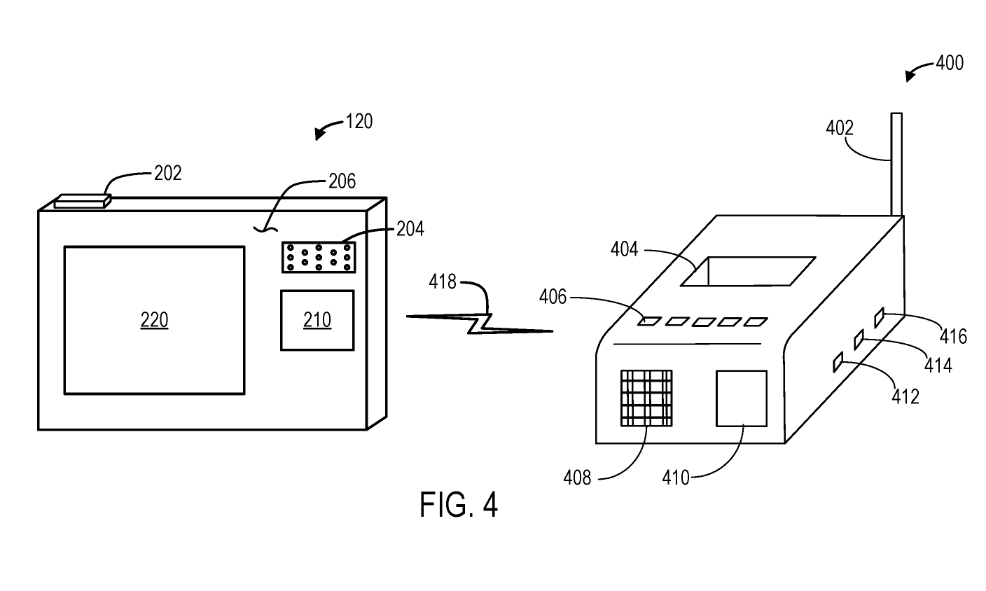

In some examples, the network interface 306 can facilitate the communication of information between the controller 120 and one or more other devices or entities over a communications network. For example, the network interface 306 may be configured to communicate with a server (e.g., a remote server 322) where a caregiver can access information related to the patient. As discussed in more detail below with reference to FIG. 4, the network interface 306 may facilitate communication between the medical device controller 120 and a base station associated (e.g., paired) with the medical device controller.

In some examples, the medical device controller 120 includes a cardiac event detector 320 to monitor the cardiac activity of the patient and identify cardiac events experienced by the patient based on received cardiac signals. In some examples, the cardiac event detector 320 can access patient templates (e.g., which may be stored in the data storage 304 as patient data 316) that can assist the cardiac event detector 320 in identifying cardiac events experienced by the particular patient.

In some implementations, the processor 318 includes one or more processors that each can perform a series of instructions that result in manipulated data and/or control the operation of the other components of the controller 120.

The controller 120 may be well-suited for a range of different cardiac monitoring and/or treatment devices and some additional examples of medical devices that incorporate the controller 120 are described further below.

In some implementations, the controller 120 comprises an embedded operating system that supplies file system and networking support. For example, such an operating system may be a Windows based operating system. In some examples, the controller 120 includes software features that provide networking security, firewalling, and data encryption services. The controller 120 can implement a data security model to prevent unauthorized access to the controller 120 and access only by authorized authenticated communication devices and/or servers. Accordingly, for any wireless transmission (e.g., Bluetooth, WiFi, or others) between the controller 120 and the garment and/or between the controller 120 and the communication device and/or server, multiple layers of security can be deployed. For example, firewall rules may be implemented in the controller 120 and/or an operating system of the communication device that will not permit external connections if they do not originate for an authorized and authenticated device. Instead of or in addition to the firewall rules described above, the controller 120 may require a unique secure login key. In some implementations, communications between any combination of the controller 120, the garment 110, the communication device, and the server may be configured to be via a Virtual Private Network (VPN). Further, in some implementations, the controller 120 can be configured to encrypt any transmitted and/or received data in compliance with prescribed security mechanisms for patient data. For example, such an encryption standard may be an AES 256 bit Cypher Block Chaining Technology.

Example Base Station

In some examples, the controller 120 may be in communication with a base station capable of performing a number of different functions. FIG. 4 shows the controller 120 in communication with a base station 400. As illustrated, the base station 400 includes an antenna 402, a battery charging bay 404, one or more buttons 406, a speaker 408, a display 410, and one or more communication interfaces 412, 414, and 416. The base station 400, in some examples, may omit one or more of the elements depicted in FIG. 4.

The base station 400 communicates with the medical device controller via, for example, a wireless communication connection 418. The wireless communication connection may be implemented through any one or combination of wireless communication standards and protocols including, for example, Bluetooth, Wireless USB, ZigBee, and Wireless Ethernet. In some examples, the controller 120 may be paired to a particular base station 400 through one or more procedures as described further below. The controller 120 may provide, for example, information regarding the patient's medical condition and/or the status of the medical device to the base station 400. The controller 120 can also provide to the base station 400 information related to the identity of the garment 100, information related to an amount of time that one or more garments have been worn, information related to the laundering of the garment 110, and information related to the state of the garment 110.

The information received by the base station 400 may be communicated over a network shortly after it is received by the base station 400, or alternatively, may be stored in a memory of the base station 400 and communicated over the network at a later time. The information that is communicated by the base station 400 may be retained in the memory of the base station 400.

The base station 400 can also store and/or communicate information received from the medical device controller 120 over a wired or wireless communication network. For example, information relating to the patient's medical condition over a period of time may be communicated by the base station 400 to a healthcare provider, such as a doctor, so that the doctor may remotely monitor the patient's medical condition. The base station 400 also includes several different communication interfaces. These communication interfaces include a device communication interface 412 to receive information from the controller 120, a telephone landline interface 414 to communicate, via a telephone network, the information received from the controller 120, and a network interface 416 to communicate, via a wired network connection (e.g., such as Ethernet), the information received from the controller 120. In some implementations, the base station 400 also includes an antenna 402 that can wirelessly communicate the information received from the controller 120 via a cellular (e.g., 2G, 3G, and 4G) network.

In some implementations, the base station 400 is capable of charging a rechargeable battery for the controller 120. In some examples, the base station 400 may include a battery charging bay 404 constructed to receive and charge a battery (e.g., the battery 212 of FIG. 2) for the controller 120.

Garment Monitoring Component

Referring again to FIG. 3, the garment monitoring component 170 is configured to interact with the processor 318 and the one or more garment monitoring sensors 324. In some implementations, the garment monitoring component 170 includes a separate processor. The garment monitoring sensors 324 can include any combination of a temperature sensor 326, a humidity sensor 328, a strain sensor 330, and a tear sensor 332, among others. The garment monitoring component 170 is configured to receive information from the garment monitoring sensors 324 to determine a condition (e.g., an environmental condition) and/or a state of the garment (110 of FIG. 1). For example, the garment monitoring component 170 can use information received from the garment monitoring sensors 324 to determine that the garment 110 is in or has been in a laundering environment. Such information can be used to infer a state of the garment 110, as described in more detail below. In some implementations, the garment monitoring component 170 can use information received from the garment monitoring sensors 324 to directly determine information related to the state of the garment 110.

One or more of the garment monitoring sensors 324 may be permanently disposed or removably disposed in the garment 110, as described in more detail below with respect to particular ones of the garment monitoring sensors 324. For example, one or more of the garment monitoring sensors 324 may be stitched into fabric of the garment 110, disposed between layers (e.g., layers of fabric) of the garment 110, disposed in a compartment of the garment 110 (e.g., a pocket sewn into the garment 110), etc. In some implementations, one or more of the garment monitoring sensors 324 can be removed from the garment 110 prior to laundering (e.g., to prevent damage to the garment monitoring sensor 324). In some implementations (e.g., implementations in which one or more of the garment monitoring sensors 324 is permanently disposed into the garment 110), one or more of the garment monitoring sensors 324 may be waterproof and/or incorporated into a waterproof compartment of the garment 110.

In some implementations, the garment monitoring component 170 is configured to determine whether the garment 110 has been laundered according to a predetermined schedule. As mentioned above, the garment 110 can temporarily lose its elasticity (e.g., stretch) after prolonged wear, which can compromise the efficacy of the sensing electrodes 112 and treatment electrodes 114. Laundering the garment 110 can restore at least some of the elasticity of the garment 110 for a variety of reasons. For example, exposing the garment 110 to moisture and subsequently drying the garment 110 can restore elasticity. In some cases, the degree of heat used for drying the garment 110 also has an impact on the amount of elasticity that is restored. For example, drying the garment 110 in a relatively warm environment (e.g., at medium to medium-high heat in a laundry dryer) can cause more elasticity to be restored to the garment 110 than what would occur if the garment 110 were air dried.

In some implementations, the garment 110 is to be laundered according to manufacturer specifications. The manufacturer specifications can call for the garment 110 being laundered using warm water, a mild clothes detergent, no bleach, and no fabric softener. The garment 110 should preferably be laundered separately from other clothes. The garment 110 is to be machine dried on a medium setting to facilitate restoration of the elasticity. In some implementations, the garment 110 is washed in a washing machine using warm water and then tumbled dry in a laundry dryer at a medium heat setting. The combination of the heat and the tumbling from the laundry dryer can facilitate restoration of elasticity in the garment 110. In some implementations, the temperature of the water used to wash the garment 110 is less than 45° C. In some implementations, the operating range of the garment 110 is approximately 0° C. to 50° C. (e.g., to prevent damage to the garment 110). In some implementations, the garment 110 should be stored in a humidity range of approximately 0 to 95% relative humidity (e.g., to prevent damage to the garment 110). Under such example conditions, the garment 110 may be configured to maintain a satisfactory elasticity for approximately six months, provided the garment 110 is periodically laundered as described above. In some examples, the six month life of the garment 110 may be based on a garment wear rotation of two garments or three garments.

The predetermined schedule according to which the garment 110 should be laundered may be based on historical information related to how long a garment can typically be worn before it develops insufficient elasticity. For example, the predetermined schedule may be based on an amount of time after which garments typically lose their elasticities due to continuous wear. If the garment 110 is not laundered according to the predetermined schedule, the garment 110 may be in (or may soon be in) a condition that in inadequate for proper functionality of the wearable medical device 100.

The garment monitoring component 170 can determine times at which the garment 110 has been laundered based on data received by the temperature sensor 326 and/or the humidity sensor 328. If the temperature sensor 326 measures a temperature value beyond a predetermined threshold and/or the humidity sensor 328 measures a humidity value beyond a predetermined threshold, the garment monitoring component 170 can determine that the garment 110 is being laundered. In some examples, the predetermined temperature threshold is approximately 100° F. to approximately 110° F., and the predetermined humidity threshold is approximately 99% Relative Humidity (RH). In some implementations, the garment monitoring component 170 determines that the garment 110 is being laundered if the measured temperature and humidity are beyond their corresponding thresholds concurrently.

In some implementations, the temperature sensor 326 includes a bimetallic strip. The bimetallic strip is a temperature-sensitive electrical contact that includes two bands of different metals joined face to face along their lengths. When the bimetallic strip is in a heated environment, the metals expand at different rates, thereby causing the strip to bend. The bimetallic strip can be tuned such that particular amounts of bend correspond to particular temperatures. The temperature sensor 326 can also include an electrical lead that is positioned such that the bimetallic strip makes contact with the lead when the predetermined threshold temperature is experienced. The contact may cause a signal to be provided to the garment monitoring component 170 indicating that the predetermined threshold temperature has been met or exceeded. In some implementations, the temperature sensor 326 includes a mechanical indicator that is positioned such that the bimetallic strip makes contact with and pushes the indicator as increased temperature is experienced. The indicator may have a plurality of assumable positions, each position corresponding to a particular temperature. For example, when the predetermined threshold temperature is experienced, the bimetallic strip may push the indicator into a position that is labeled “Laundering Temperature Achieved,” thereby providing a visual indication on the garment 110 that the garment 110 has experienced a temperature that corresponds to a laundering environment.

The temperature and humidity measurements made by the temperature sensor 326 and the humidity sensor 328 can be stored along with corresponding timing information in an event log. For example, when a temperature and a humidity that exceeds the corresponding predetermined thresholds is detected by the garment monitoring component 170, a laundering event can be identified and stored along with the time at which the laundering event occurred. In some implementations, temperatures and humidities are stored in the event log according to a predetermined interval (e.g., even if a laundering event is not identified). The event log can be stored on a memory module of the garment monitoring component 170. The garment monitoring component 170 may provide at least some of the information stored in the event log to the controller 120 according to a predetermined interval (e.g., every minute, every five minutes, every hour, etc.).

The controller 120 can provide information related to the laundering of the garment via the user interface 308 (e.g., including the touch screen 220 of FIG. 2). For example, after the garment monitoring component 170 determines that a laundering event has occurred and information related to the determination is provided to the controller 120, the controller 120 can provide a message on the touch screen 220 indicating such (e.g., “Garment Laundering Detected”). Similarly, if a laundering event has not been detected by the garment monitoring component 170 for a particular amount of time, the controller 120 can provide a message indicating such (e.g., “Garment Has Not Been Laundered for X Days”). The message serves to remind the patient that garment laundering is overdue.

In some implementations, the garment monitoring sensors 324 can include a pH sensor for measuring a pH level of moisture. The garment monitoring component 170 can consider the measurements from the pH sensor when determining whether the garment 110 is in a laundering environment.

In some implementations, the garment monitoring sensors 324 include a strain sensor 330 for measuring strain values (e.g., stretch values) of the garment 110. The stretch values indicate the elasticity of the garment 110. The garment monitoring component 170 can consider the measurements from the strain sensor 330 when determining whether the garment 110 is in a laundering environment. For example, if the stretch values do not meet or exceed a predetermined stretch threshold over a period of time, the garment monitoring component 170 can determine that the garment 110 was not laundered over the period of time. In some implementations, the garment monitoring component 170 considers multiple stretch values to determine a change of stretch (e.g., a stretch delta) of the garment 110 when determining whether the garment 110 has been in a laundering environment. For example, the garment monitoring component 170 can determine that the garment 110 was in a laundering environment if a difference between a first stretch value measured at a first time and a second stretch value measured at a second time meets or exceeds a predetermined threshold. Such an approach can account for relative stretch reduction, rather than absolute stretch reduction, which can be a more accurate indicator of the garment 110 being laundered.

As described above, the garment monitoring component 170 determines whether the garment 110 has been laundered according to a predetermined schedule. The predetermined scheduled can be based on historical information related to how long a garment can typically be worn before it develops insufficient elasticity. Thus, an inference can be made as to the state of the garment 110 based on occurrences of laundering events experienced by the garment 110. However, in some implementations, the garment monitoring component 170 can use information received from the garment monitoring sensors 324 to directly determine information related to the state of the garment 110.

As previously mentioned, the garment monitoring sensors 324 can include a strain sensor 330 (sometimes referred to as a strain gauge). The strain sensor 330 is configured to measure a strain (e.g., a stretch) experienced by the garment 110. In some implementations, the strain sensor 330 includes one or more coils configured to provide information related to a magnitude of stretch experienced by the garment 110 to the garment monitoring component 170. The strain sensor 330 can include one or both of an inductive element and a capacitive element.

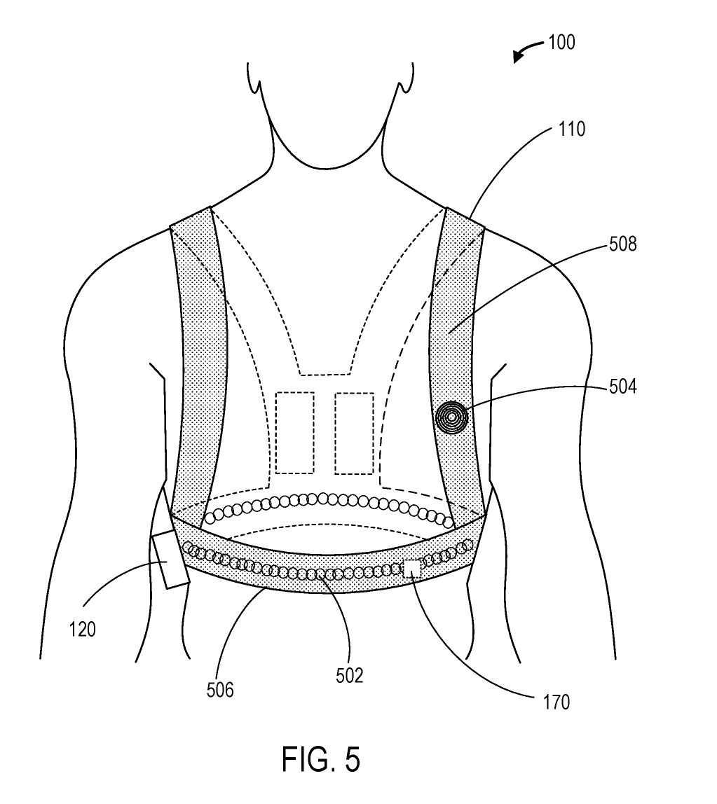

FIG. 5 shows an example of the wearable medical device 100 of FIG. 1 that includes a strain sensor. The strain sensor includes an inductive element 502 and a capacitive element 504. The inductive element 502 can include a coil embedded in fabric of the garment 110. In particular, the coil can be embedded in a belt portion 506 of the garment 110. As shown in FIG. 5, the inductive element 502 can include a metallic or other conductive material, such as a conductive wire or thread that is configured in a coiled pattern (e.g., spring shaped) that includes a plurality of windings (or turns) with a substantially similar radius. In some implementations, a magnetic core may be disposed within the windings of the inductive element 502. In some implementations, the inductive element 502 can be configured in a serpentine pattern that includes a plurality of serpentine turns of similar dimension.

The capacitive element 504 can also include a coil embedded in fabric of the garment 100. In particular, the coil can be embedded in a strap portion 508 of the garment 110. The capacitive element 504 can include a conductive material, such as a pair of wires having a round circumference or at least one flat side, configured in parallel with each other in a coiled or serpentine pattern. In some implementations, the capacitive element 504 includes two wires with a space therebetween. The space can be occupied by an insulator, a dielectric material, or air such that the two wires of the capacitive element 504 act as a capacitor when supplied with power.

The inductive element 502 and the capacitive element 504 can be shielded, jacketed, or insulated, and can be configured to connect to a power supply. For example, the inductive element 502 and the capacitive element 504 can be powered by the battery (310 of FIG. 3) of the controller 120.

The inductive element 502 and the capacitive element 504 are configured to stretch or expand together with the garment 110. For example, the inductive element 502 can include a coil with a particular number of turns over a defined length. The radius of the coil changes when the inductive element 502 is stretched (e.g., when the belt portion 506 garment 110 is stretched). When the radius of the coil changes the inductance of the inductive element 502 also changes. Similarly, when the capacitive element 504 is stretched, the measured capacitance changes. One or both of the inductive element 502 and the capacitive element 504 can provide a measured value (e.g., an inductance value and a capacitance value, respectively) indicative of a magnitude of stretch experienced by the garment 110 to the garment monitoring component 170. In this way, the inductance value and the capacitance value act as stretch values. If one or both of the inductance value and the capacitance value meets or exceeds a predetermined threshold, the garment monitoring component 170 can determine that the state of the garment 110 is unsatisfactory (and, e.g., cause an alert to be provided to the patient indicating such). In particular, the garment monitoring component 170 can determine that the garment 110 has inadequate elasticity for ensuring sufficient contact between the patient and the electrodes. For example, an inductance value and/or a capacitance value outside of a tolerance range or above or below a threshold can indicate that the sensing electrodes 112 and/or the therapy electrodes 114 are improperly positioned or tensioned due to stretching of the garment 110, and an inductance value and/or a capacitance value within a tolerance range or above or below a threshold can indicate that no or minimal stretching of the garment 110 has occurred and that the sensing electrodes 112 and/or the therapy electrodes 114 are properly positioned on the patient. In some implementations, the predetermined threshold is related to a baseline inductance and/or capacitance values measured by the inductive element 502 and/or the capacitive element 504 when the garment 110 is not being worn. In some implementations, the predetermined threshold is determined by the manufacturer.

If the state of the garment 110 is unsatisfactory (e.g., if the garment 110 has an elasticity insufficient for ensuring sufficient contact between the patient and the electrodes), the garment 110 may require laundering in order to restore the garment 110 to a satisfactory state. For example, if the garment 110 has temporarily developed insufficient elasticity due to prolonged use between launderings, then laundering the garment 110 can restore the garment 110 to a satisfactory condition. As described in more detail below, in some implementations, the garment laundering component 170 can be configured to alert the patient to replace the garment 110 with another garment and/or launder the garment 110 under such circumstances.

In some implementations, the sensing electrodes 112 and/or the therapy electrodes 114 are properly positioned when they are adequately pressured by the garment 110 to be firmly pressed against the patient such that the electrodes 112, 114 are held in a substantially fixed position to sense the patient's condition or deliver treatment. Properly positioned electrodes, 112, 114 are generally held by the garment 110 in a fixed position with minimal movement and in contact with the patient's skin.

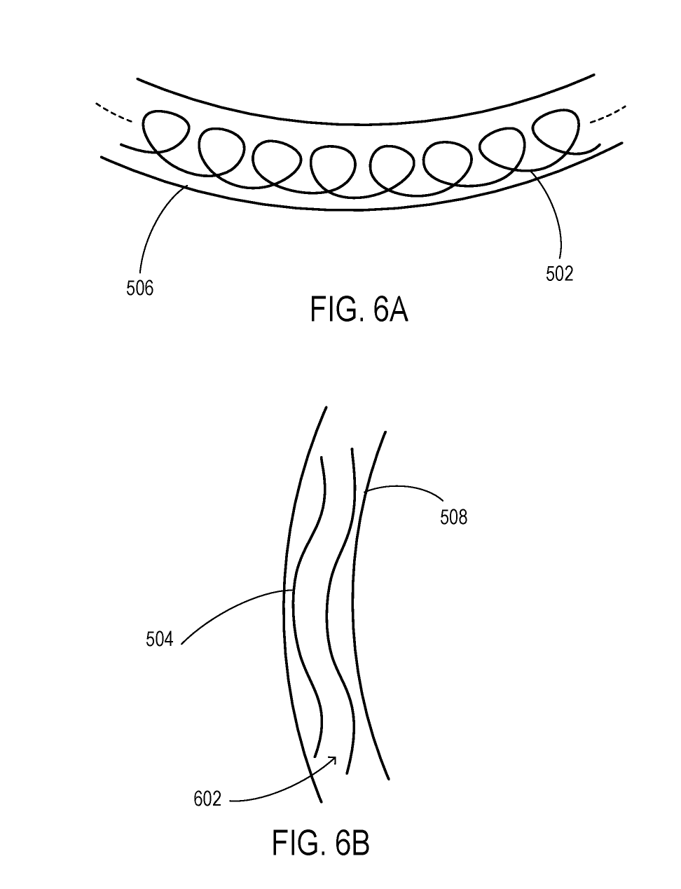

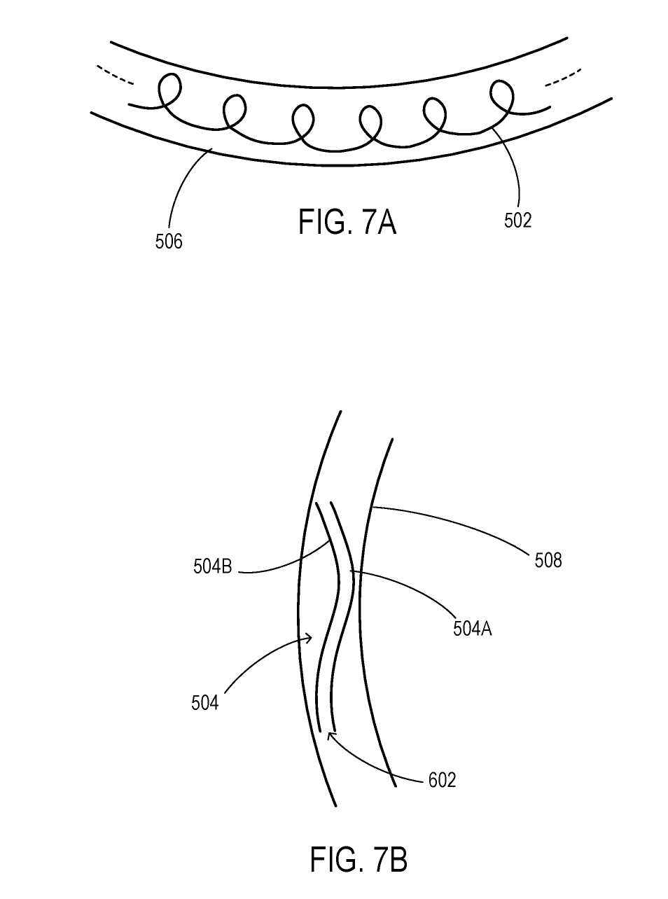

FIG. 6A shows an example of the inductive element 502 of FIG. 5 in an unstretched position (e.g., when the belt portion 506 of the garment 110 is in an unstretched position), and FIG. 7A shows an example of the inductive element 502 in a stretched position (e.g., when the belt portion 506 of the garment 110 is in a stretched position). The garment monitoring component 170 can determine differences between inductance measurements provided by the inductive element 502 in the unstretched position shown in FIG. 6A and the stretched position shown in FIG. 7A. For example, the garment 110 may have the unstretched configuration of FIG. 6A when the garment 110 has an acceptable elasticity, and may have the stretched configuration of FIG. 7A if the garment has an unacceptable elasticity (e.g., due to excessive continuous wear). While the inductive element 502 is shown as being positioned in the belt portion 506 of the garment 110, the inductive element 502 can be positioned elsewhere, such as in the strap portion 508 of the garment 110.

FIG. 6B shows an example of the capacitive element 504 of FIG. 5 in an unstretched position (e.g., when the strap portion 508 of the garment 110 is in an unstretched position), and FIG. 7B shows an example of the capacitive element 504 in a stretched position (e.g., when the strap portions 508 of the garment 110 is in a stretched position). A space 602 may be present between two portions (e.g., wires 504A, 504B) of the capacitive element 504. The garment monitoring component 170 can determine differences between capacitance measurements provided by the capacitive element 504 in the unstretched position shown in FIG. 6B and the stretched position shown in FIG. 7B, as stretching of the garment 110 changes the relative capacitive surface area and/or distance between the wires 504A, 504B. While the capacitive element 504 is shown as being positioned in the strap portion 508 of the garment 110, the capacitive element 504 can be positioned elsewhere, such as in the belt portion 506 of the garment 110.

In some implementations, the inductive element 502 can have a shape other than a coil shape. For example, the inductive element 502 may have a serpentine shape similar to the shape of the wires 504A, 504B of the capacitive element 504 shown in FIGS. 6B and 7B.

The inductive element 502 and/or the capacitive element can include a wire or thread sewn into the garment 110. In some implementations, the inductive element 502 and/or the capacitive element 504 can expand or contract with the garment 110, but otherwise substantially maintain their shapes. In some implementations, the inductive element 502 can be wrapped around a core included in the garment 110.

The garment monitoring sensors 324 can also include a tear sensor 332. The tear sensor 332 is configured to measure one or more electrical properties of a portion of the garment 110 to determine whether a tear (e.g., a rip) is present in the garment 110. A tear in the garment 110 can indicate that the garment 110 is in a deteriorated state, and may be indicative of an unacceptable degree of stretch in the garment 110. As explained above, this may result in one or more of the electrodes 112, 114 being improperly positioned on the patient or being in insufficient contact with the patient. In some implementations, even if the garment 110 is experiencing an acceptable degree of stretch as indicated by the strain sensor 330, the presence of a tear based on measurements made by the tear sensor 332 may indicate that the garment 110 will soon be in an unacceptable state. Accordingly, the garment monitoring component 170 may determine that the garment should be replaced based on measurements made by the tear sensor 332 irrespective of information received from the other garment monitoring sensors 324. The tear sensor 332 can include one or more conductive meshes.

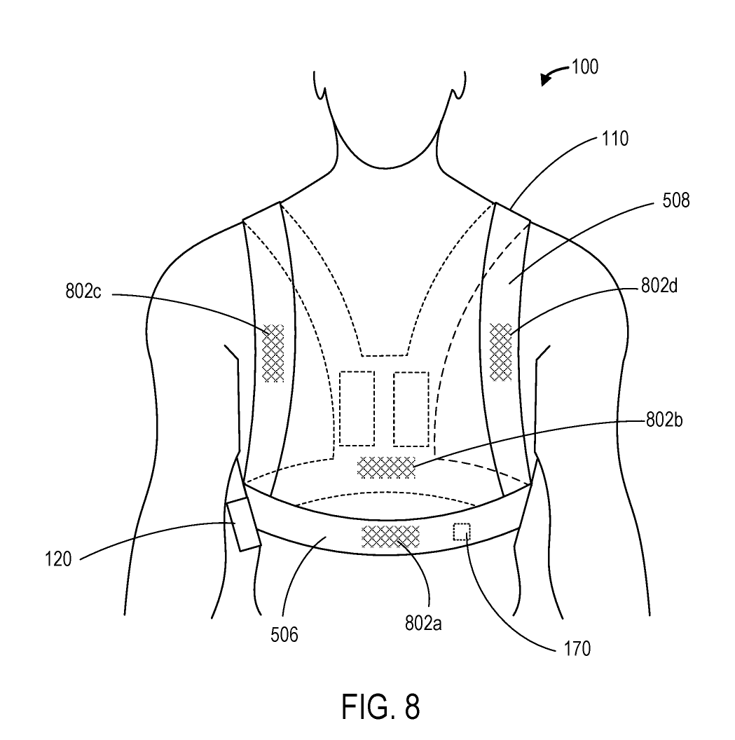

FIG. 8 shows an example of the wearable medical device 100 of FIG. 1 that includes a tear sensor. The tear sensor includes a plurality of conductive meshes 802a-d. The conductive meshes 802a-d are embedded in fabric of the garment 110 in various portions of the garment. In this example, first and second conductive meshes 802a, 802b are embedded in the fabric of the belt portion 506 of the garment 110, and third and fourth conductive meshes 802c, 802d are embedded in the strap portion 508 of the garment 110. In some implementations, the garment 110 includes fewer or additional conductive meshes. In some implementations, the garment 110 includes a single conductive mesh that is embedded in a relatively large portion of the garment 110. For example, the belt portion 506 of the garment 110 can include a single conductive mesh that is positioned around the circumference of the belt portion 506. In some implementations, the fabric of the garment 110 itself is made of a conductive material that is arranged as a conductive mesh. In this way, a substantial portion of the garment 110 (e.g., the entire garment 110) can be monitored for tears. In some implementations, tear sensors may be implemented in one or more portions of the garment that is more susceptible to strain, wear, and/or tear than other portions. For instance, the tear sensor may be implemented proximate the rear of the garment along the belt dimension.

The conductive meshes 802a-d include a metallic or other conductive material, such as a conductive wire or thread. The conductive meshes 802a-d can be shielded, jacketed, or insulated, and can be configured to connect to a power supply. For example, the conductive meshes 802a-d can be powered by the battery (310 of FIG. 3) of the controller 120. The conductive meshes 802a-d can be characterized as having a particular resistive value (e.g., in ohms). When the conductive meshes 802a-d are in a like-new condition, they may have a relatively low resistance. Over time, the resistance of the conductive meshes 802a-d can decrease for a number of reasons. As the conductive materials of the conductive meshes 802a-d wear down and/or oxidize, their resistances can increase. Further, the presence of tears in the conductive meshes 802a-d can cause their resistances to increase, thereby allowing less current to travel therethrough.

Each of the conductive meshes 802a-d can provide measured values related to one or more electrical properties (e.g., current values and/or resistance values and/or voltage values) to the garment monitoring component 170. For example, a predetermined voltage may be applied across each conductive mesh 802a-d by a power supply, thereby causing current to travel through each conductive mesh 802a-d. The amount of current that travels through each conductive mesh 802a-d when a particular voltage is applied depends on the resistance of the conductive mesh 802a-d. The predetermined voltage may be provided to the garment monitoring component 170 or the predetermined voltage may be previously known to the garment monitoring component 170.

The resultant current value can be provided to the garment monitoring component 170, and using Ohm's Law, the resistance of the conductive meshes 802a-d can be determined. If a resistance value of a conductive mesh 802a-d meets or exceeds a predetermined threshold, the garment monitoring component 170 can determine that the state of the garment 110 is unacceptable. In particular, based on the resistance values, the garment monitoring component 170 can determine that a tear is present in one or more of the conductive meshes 802a-d. In some implementations, the garment monitoring component 170 can determine that a tear is not yet present in one of the conductive meshes 802a-d, but that the resistance value indicates a deteriorated condition of the conductive mesh 802a-d that may soon result in a tear developing in the conductive mesh 802a-d. In either case, a resistance value that meets or exceeds the predetermined threshold can indicate that the garment 110 has reached or is about to reach its end of life and should be replaced. In some implementations, the predetermined threshold is related to a baseline resistance value of the conductive meshes 802a-d taken when they are in new condition. In some implementations, the predetermined threshold is determined by the manufacturer.

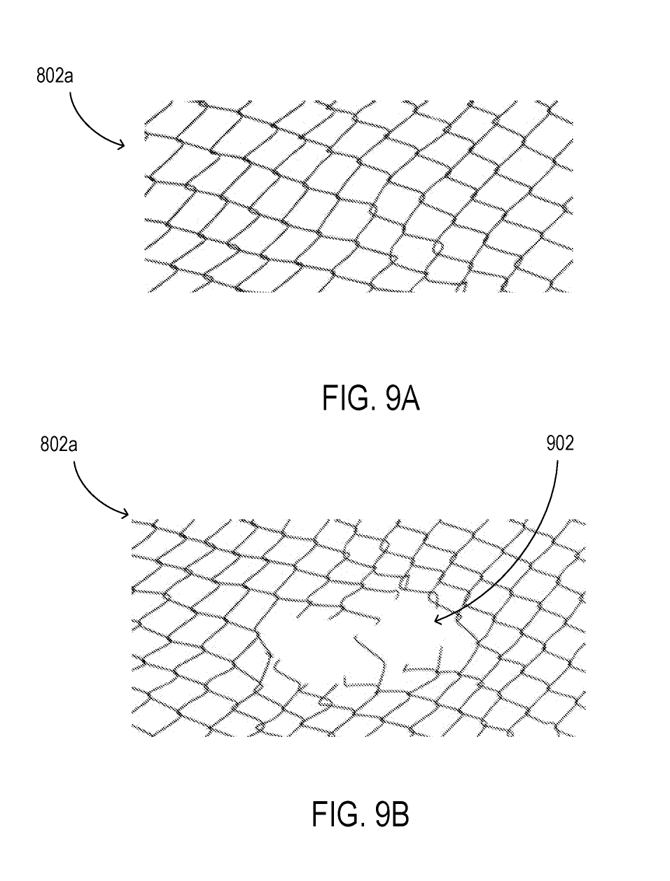

FIG. 9A shows an example of one of the conductive meshes 802a of FIG. 8 in a like-new condition, and FIG. 9B shows an example of the conductive mesh 802a after it has developed a tear 902. For example, the conductive mesh 802a of FIG. 9B may have developed the tear 902 after a prolonged period of use by the patient (e.g., weeks, months, or even years). The garment monitoring component 170 can determine differences between current and/or resistance measurements of the conductive mesh 802a in the like-new condition shown in FIG. 9A and current and/or resistance measurements of the conductive mesh 802a in the deteriorated condition shown in FIG. 9B. For example, a relatively small voltage (e.g., 100 mV) may be applied across the conductive mesh 802a. In like-new condition, the conductive mesh 802a may have a relatively small resistance (e.g., 5 ohms). Thus, the resultant current that travels through the like-new conductive mesh 802a of FIG. 9A would be 20 mA. The garment monitoring component 170 can determine the resistance of the like-new conductive mesh 802a based on the voltage and current values. The garment monitoring component 170 can compare the determined resistance value to a predetermined threshold to determine that the conductive mesh 802a of FIG. 9A is in like-new condition.

As the conductive mesh 802a of FIG. 9A deteriorates over time (e.g., due in part to excessive wear of the garment 110), it may develop a tear 902 as shown in FIG. 9B. In the deteriorated state, the torn conductive mesh 802a may have a higher resistance (e.g., 20 ohms) as compared to the like-new conductive mesh 802a of FIG. 9A. Thus, the resultant current that travels through the deteriorated conductive mesh 802a of FIG. 9B would be 5 mA. The garment monitoring component 170 can determine the resistance of the deteriorated conductive mesh 802a based on the voltage and current values. The garment monitoring component 170 can compare the determined resistance value to a predetermined threshold to determine that the conductive mesh 802a of FIG. 9A is in a deteriorated condition. In this example, the predetermined threshold may be approximately 10 ohms. The predetermined threshold may be chosen such that a conductive mesh 802a-d is identified as being in a deteriorated condition (e.g., due to deterioration and/or oxidation of the conductive material) before a full tear has developed in the conductive mesh 802a-d.

Referring to FIG. 10, the garment monitoring component 170 is configured to receive information from one or more of the garment monitoring sensors 324 to determine the state of the garment 110. The garment monitoring sensors 324 can include the temperature sensor 326, the humidity sensor 328, the strain sensor 330, and the tear sensor 332, although other sensors can also be included.

While the garment monitoring component 170 has been largely described as being incorporated into the garment 110, in some implementations, the garment monitoring component 170 may be positioned elsewhere on the medical device (e.g., the wearable medical device 100). For example, in some implementations, the garment monitoring component 170 may be incorporated into the medical device controller 120. In some implementations, the garment monitoring component 170 may be incorporated as part of the processor (318 of FIG. 3). In some implementations, the processor 318 may be configured to perform the functions of the garment monitoring component 170 such that a separate chip (e.g., a separate processor) is not required.

The garment monitoring component 170 can directly determine the state of the garment 110 based on data received by any combination of the garment monitoring sensors 324. For example, stretch values received from the strain sensor 330 alone (e.g., stretch values that meet or exceed a predetermined threshold) can be used to determine that the garment 110 is in an unsatisfactory condition; resistance values received by the tear sensor 332 alone (e.g., resistance values based on voltage and current values that meet or exceed a predetermined threshold) can be used to determine that the garment is in an unsatisfactory condition. In some implementations, a combination of conditions must be satisfied for the garment monitoring component 170 to determine that the garment 110 is in an unsatisfactory condition. For example, both a stretch value that meets or exceeds a predetermined threshold and a resistance value that meets or exceeds a predetermined threshold may need to be received by the garment monitoring component 170 for the garment monitoring component 170 to determine that the garment 110 is in an unsatisfactory condition.

While the temperature sensor 326 and the humidity sensor 328 have largely been described as being used to determine whether the garment 110 has been in a laundering environment, data received from the temperature sensor 326 and the humidity sensor 328 can also be used by the garment monitoring component 170 to directly determine the state of the garment 110. For example, the garment monitoring component 170 can consider temperature values and humidity values received over a period of time to determine the types of environments that the garment 110 has been exposed to and the frequency of exposure to such environments. Excessive exposure to high or low temperatures and/or high or low humidities can cause the garment 110 to degrade. If the received values correspond to repeated exposure to an environment that is typically harmful to the garment (e.g., excessive exposure to heat and/or moisture), the garment monitoring component 170 may determine that the state of the garment is unsatisfactory.

In some implementations, information received from the temperature sensor 326 and/or the humidity sensor 328 can be used in combination with information received from the other garment monitoring sensors 324 to determine the state of the garment 110. For example, stretch values received from the strain sensor 330 and/or resistance values received from the tear sensor 332 may almost, but not quite meet or exceed the corresponding predetermined thresholds. If the temperature values and the humidity values indicate that the garment 110 has excessively been exposed to harmful conditions, the garment monitoring component 170 may relax (e.g., lower) the predetermined thresholds such that the stretch values and the resistance values meet or surpass the relaxed thresholds, thereby resulting in a determination that the garment 110 is in an unsatisfactory condition.

The garment monitoring component 170 can include one or both of a processor and a memory module for storing data received from the garment monitoring sensors 324. In some implementations, the garment monitoring component 170 interacts with another processor of the wearable medical device 100 (e.g., the processor 318 of the controller 120).

The garment monitoring component 170 can store information received from the garment monitoring sensors 324 in an event log 1000. The event log 1000 can store the values measured by the garment monitoring sensors 324, including temperature values, humidity values, stretch values, and current and/or resistance values. The values can be stored along with timing information that corresponds to times at which the measurements occurred. In this way, the event log 1000 can include historical data related to the garment that spans over a period of time.

The event log 1000 can be stored on a memory module of the garment monitoring component 170. In some implementations, data collected by the garment monitoring sensors 324 is stored on memory module of the corresponding garment monitoring sensors 324, and the data is provided to the garment monitoring component 170 (e.g., via a wireless or wired connection) and stored in the event log 1000. Information stored in the event log 1000 is then provided to the controller 120. In some implementations, the event log 1000 is stored on the controller 120 (e.g., in the data storage 304 of FIG. 3), and data collected by the garment monitoring sensors 324 is sent directly to the controller 120 via a wireless or wired connection.

The data collected by the garment monitoring sensors 324 can be provided by the garment monitoring component 170 to the event log 1000 according to a predetermined interval. For example, the data can be provided to the event log 1000 every minute, every five minutes, every hour, etc. In some implementations, the data collected by the garment monitoring sensors 324 can be provided to the event log 1000 upon the occurrence of a particular event. For example, data collected by the garment monitoring sensors 324 may be provided to the event log 1000 when a measured value meets or exceeds a corresponding predetermined threshold. Measured values that meet or exceed a predetermined threshold are typically of particular interest. Thus, the event log 1000 can be populated with such relatively important data as soon as or shortly after it becomes available. Similarly, the controller 120 can receive the data collected by the garment monitoring sensors 324 according to a predetermined interval or upon the occurrence of an event. For example, the garment monitoring sensors 324 (e.g., through the garment monitoring component 170 and/or the event log 1000) can provide information to the controller 120 when a measured value meets or exceeds a predetermined threshold. In some implementations, the garment monitoring component 170 provides at least some of the information included in the event log 1000 when the controller 120 is in communication with the garment monitoring component (e.g., when the garment monitoring component 170 is within wireless transmission range of the controller 120 or when the garment monitoring component 170 is electrically connected to the controller 120).

As described above, in some implementations, the event log 1000 includes identification information related to garments (e.g., multiple garments) that have been associated with the controller 120. The identification information can include one or more values. The one or more values can include data that corresponds to a serial number associated with each garment, a model number of each garment, and a size of each garment, among other information. The event log 1000 can also include timing information (e.g., timestamps) that represent times at which particular garments became associated/disassociated with the controller 120. The garment monitoring component 170 can process the identification and timing information to determine an amount of time that each garment has been associated with the controller 120. Using this information, the garment monitoring component 170 can infer that a particular garment has been worn for a particular amount of time. Thus, when a garment has been associated with the controller 120 for a predetermined threshold amount of time (e.g., one or two days) and/or when a garment has not been removed on at least a predetermined interval, the garment monitoring component 170 may determine that the garment should be removed or replaced (e.g., with another garment). The patient can launder the previously-worn garment (e.g., to restore it to a like-new condition).

In some implementations, the event log 1000 includes temperature and humidity measurements and timestamps that correspond to each temperature and humidity measurement. The garment monitoring component 170 can process the measurements to determine times at which the garment 110 was laundered. In particular, as described above, the garment monitoring component 170 can identify temperature and/or humidity measurements that meet or exceed corresponding predetermined temperature and humidity thresholds and determine that the garment 110 was laundered at a time as indicated by the associated timestamp. Using this information, the garment monitoring component 170 can determine whether the garment 110 has been laundered according to a predetermined threshold.

In some implementations, the event log 1000 includes stretch measurements from the strain sensor 330 and/or current/resistance measurements from the tear sensor 332. The stretch measurements and the current/resistance measurements can each include timestamps. The garment monitoring component 170 can process the measurements to determine times at which the garment 110 experienced an unacceptable stretch or tear. In some implementations, stretch measurements that occur at times during which a laundering event is identified can be used to determine the efficacy of the laundering on the garment 110. For example, the garment monitoring component 170 can monitor stretch values throughout a laundering event and compare the stretch values to temperature and humidity values that occur throughout the laundering event to identify the effect of particular laundering conditions on the garment 110. Using this information, particular temperatures and/or humidities can be identified that are effective for restoring the elasticity of the garment 110 to an acceptable state. For example, the measurement values stored in the event log 1000 may be used to determine that drying the garment 110 at 115° F. restores the garment to 70% of its original or baseline elasticity, while drying the garment 110 at 140° F. restores the garment to close to 100% of its original or baseline elasticity.

The data stored in the event log 1000 can be used to determine whether the garment 110 is reaching its end of life and thus should be discarded. For example, the event log 1000 may indicate that the garment 110 does not sufficiently restore to its original or baseline elasticity when it is laundered irrespective of the conditions of the laundering environment, or the event log 1000 may indicate that the garment 110 loses its elasticity more quickly between launderings than it did previously. The inability of the garment 110 to sufficiently restore and/or maintain its elasticity may indicate that the garment 110 has been permanently stretched or is no longer able to sustain a particular stretch (e.g., a particular tightness about the patient) for a sufficient amount of time between launderings. In such cases, the garment 110 should be discarded and replaced with a new garment.

In some implementations, the wearable medical device 100 can include additional components for indicating the state of the garment 110. In some implementations, the garment 110 can include one or more visual indicators for providing information related to the state of the garment 110. In some implementations, one or more of the visual indicators does not include electronic components.

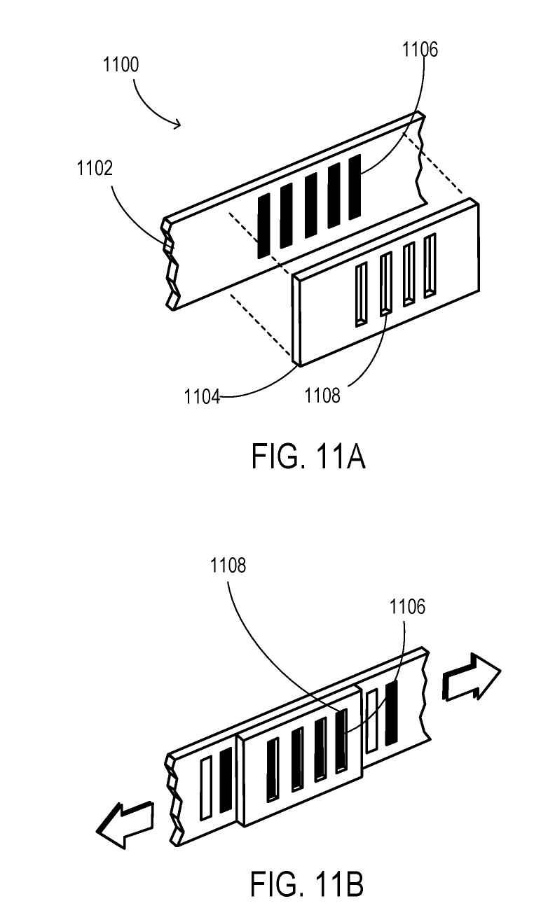

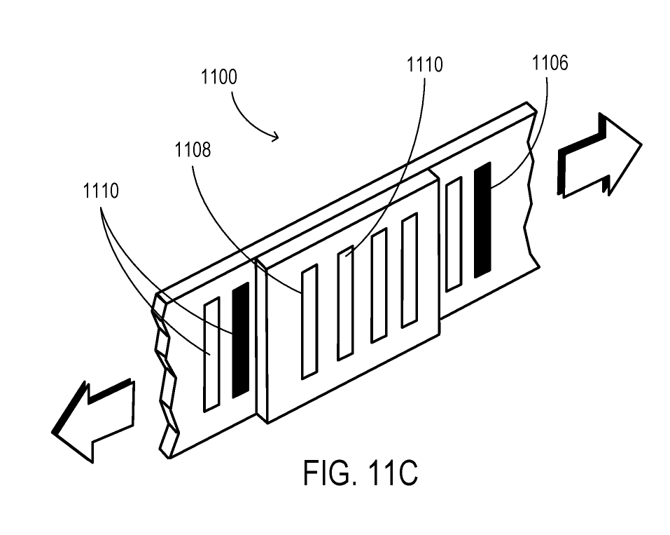

FIGS. 11A-11C show an example of a visual stretch detector 1100 that includes a garment portion 1102 and a window portion 1104. The garment portion 1102 can embedded into the garment (e.g., in the belt portion 506 or the strap portion 508 of the garment 110). The window portion 1104 can be sewn into the garment such that the window portion 1104 overlays the garment portion 1102. The garment portion 1102 includes imprints 1106 that align with corresponding slotted windows 1108 of the window portion 1104. The imprints 1106 and the windows 1108 are arranged such that the imprints 1106 are visible through the windows 1108 when the garment sufficiently fits the patient. The imprints 1106 can have a different color that the rest of the garment portion 1102 to enhance visibility. In some implementations, the imprints 1106 and the windows 1108 each have substantially similar dimensions.

In some implementations, the garment portion 1102 stretches and contracts along with the garment, while the window portion 1104 substantially retains its dimensions. In this way, the portion of the garment that is visible through the windows 1106 is indicative of a magnitude of stretch experienced by the garment. For example, when the garment has an acceptable elasticity and sufficiently fits the patient, the imprints 1106 may be entirely visible through the windows 1106, as shown in FIG. 11B. The patient can observe that the windows 1104 are completely filled by the imprints 1106 and conclude that the garment has an acceptable fit.

As the garment loses its elasticity over time (e.g., between launderings), the garment portion 1102 stretches along with the garment, resulting in a change of position of the imprints 1106. The window portion 1104 may not stretch along with the garment portion 1102, resulting in the imprints 1106 no longer being aligned with the windows 1108. For example, when the garment does not have an acceptable elasticity and does not sufficiently fit the patient (e.g., because it is overstretched), the imprints 1106 may be only partially visible or not visible through the windows 1108, as shown in FIG. 11C. Rather, non-imprint portions 1110 of the garment that reside in between or adjacent to the windows 1108 may instead be visible. The patient can observe that the windows 1108 are not filled or are partially filled by the imprints 1106 and conclude that the garment does not have an acceptable fit. In some implementations, the imprints 1106 are green and the non-imprint portions 1110 of the garment are red so that the patient can easily notice when the garment does not have an acceptable fit.

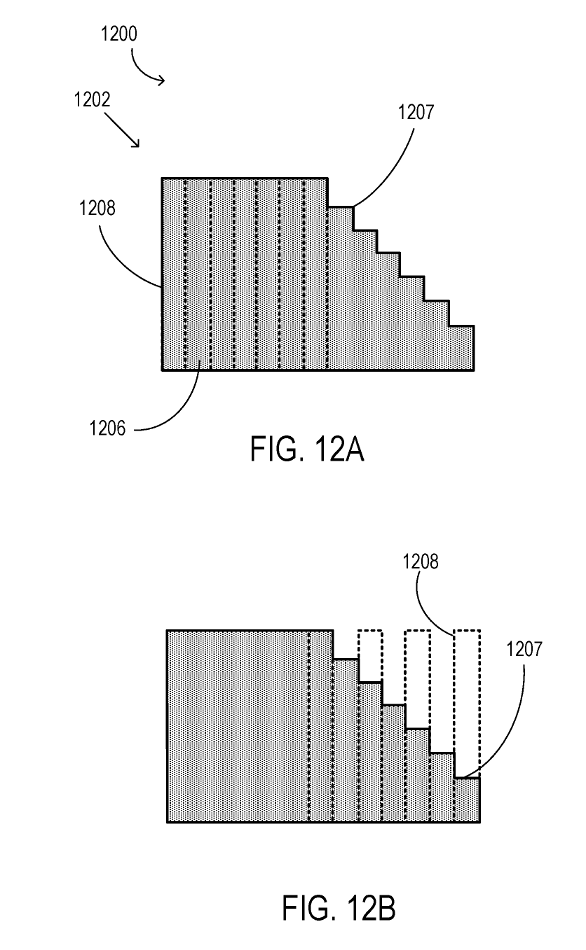

In some implementations, the amount of the imprints (e.g., by area) that is visible through the windows may indicate the magnitude of stretch experienced by the garment. FIGS. 12A-12B show another example of a visual stretch detector 1200 that includes a garment portion 1202 and a window portion that includes slotted windows 1208. The window portion of the visual stretch detector 1200 is not explicitly shown in the figure. The garment portion 1202 includes an imprint 1206 that includes a plurality of steps 1207. The imprint 1206 and the windows 1208 are arranged such that the steps 1207 are not visible through the windows 1208 when the garment sufficiently fits the patient, as shown in FIG. 12A.

As the garment loses its elasticity over time, the garment portion 1202 stretches along with the garment, resulting in a change of position of the imprint 1206. The window portion may not stretch along with the garment portion 1202, resulting in some of the steps 1207 being visible through the windows 1208, as shown in FIG. 12B. In this example, three of the steps 1207 are visible through the windows 1208.

In some implementations, the number of steps 1207 that are visible through the windows 1208 indicates the magnitude of stretch experienced by the garment. Thus, the number of steps 1207 that are visible through the windows 1208 can be used to determine whether the garment has an acceptable fit. For example, if no steps 1207 are visible through the windows 1208 (e.g., as shown in FIG. 12A), a determination can be made that the garment is in like-new condition; if one step 1207 is visible through the windows 1208, a determination can be made that the garment currently has an acceptable fit, but the garment is beginning to experience signs of excessive continuous wear; if two steps 1207 are visible through the windows, a determination can be made that the garment currently has an acceptable fit, but the garment should be laundered or replaced soon; if three steps 1207 are visible through the windows 1208 (e.g., as shown in FIG. 12B), a determination can be made that the garment no longer has an acceptable fit, and the garment should be laundered or replaced as soon as possible; if four steps 1207 are visible through the windows 1208, a determination can be made that the garment is in a state that is insufficient for proper operation of the medical device and should be laundered or replaced immediately.

In some implementations, the visual stretch detector 1100, 1200 can include additional components and/or circuitry for conveying information to the garment monitoring component 170. In some implementations, the visual stretch detector 1100, 1200 is included as one of the garment monitoring sensors (324 of FIG. 3). The visual stretch detector 1100, 1200 can provide information related to the state of the garment that can be stored in the event log (1000 of FIG. 10).

In some implementations, the visual stretch detector 1100, 1200 includes an optical sensor that is configured to capture an image of the portions of the garment (e.g., the imprints 1106, 1206) that are visible through the windows 1108, 1208. The optical sensor can provide information related to the captured image to the garment monitoring component 170. The information related to the image can include one or more values that represent a color composition of the image. For example, referring to FIG. 11B, the optical sensor may capture an image of the markings 1106 that are visible through the windows 1108. In this example, the markings 1106 are fully visible through the windows 1108; that is, no other portions of the garment portion 1202 are visible through the windows 1108. Thus, the image includes four areas that each correspond to one of the windows 1108, and each of the areas include a single color (e.g., the color of the markings 1106). The optical sensor can provide a value to the garment monitoring component 170 that represents a color composition of the image. The value may be in percentage form. In this example, the value is 100%, which signifies that the entirety of the portions of the garment that are visible through the windows 1108 include the markings 1106.

In another example, referring to FIG. 11C, the optical sensor may capture an image of the portions of the garment that are visible through the windows 1108. In this example, the markings 1106 are not at all visible through the windows 1108. Thus, the image includes four areas that each correspond to one of the windows 1108, and each of the areas include a single color (e.g., the color of the non-imprint portions 1110 of the garment). The optical sensor can provide a value to the garment monitoring component 170 that represents a color composition of the image. The value may be in percentage form. In this example, the value is 100%, which signifies that the entirety of the portions of the garment that are visible through the windows 1108 do not include the markings 1106.

In another example, referring to FIG. 12B, the optical sensor may capture an image of the portions of the garment that are visible through the windows 1208. In this example, the marking 1206, in particular the steps 1207 of the marking 1206, is partially visible through the windows 1208. Thus, the image includes four areas that each correspond to one of the windows 1208. A first area includes a single color (e.g., the color of the marking 1206); a second area includes approximately 75% the color of the marking 1206 and approximately 25% the color of the non-imprint portion of the garment; a third area includes approximately 50% each of the color of the marking 1206 and the color of the non-imprint portion of the garment; and a fourth area includes approximately 25% the color of the marking 1206 and approximately 75% the color of the non-imprint portion of the garment. In total, the image includes approximately 60% the color of the marking 1206 and approximately 40% the color of the non-imprint portion of the garment. The optical sensor can provide a value to the garment monitoring component 170 that represents a color composition of the image. In this example, the value is 60%, which signifies that 60% of the portions of the garment that are visible through the windows 1208 include the marking 1206.

The garment monitoring component 170 can determine a magnitude of the elasticity of the garment based on the value that represents the color composition of the image. For example, the visual stretch detector 1100, 1200 can be calibrated such that particular color compositions correspond to particular stretch values experienced by the garment. In some implementations, the garment monitoring component 170 can compare the value that represents the color composition of the image to one or more predetermined threshold values to determine whether the garment is in an unsatisfactory condition. In some implementations, the predetermined threshold value may be 50% (e.g., 50% of the portions of the garment that are visible through the windows 1108, 1208 include the marking 1106, 1206). Thus, if the color composition of the image includes less than 50% of the color of the markings 1106, 1206, the garment monitoring component 170 can determine that that garment is in an unsatisfactory condition and should be laundered or replaced.

In some implementations, the visual stretch detector 1100, 1200 can include conductive strips and conductivity sensors. The conductive strips can be incorporated into an outside surface of the garment portion 1102, 1202 and the conductivity sensors can be incorporated into an inside surface of the window portion 1104. In some implementations, first conductive strips are incorporated into the imprints 1106, 1206 and second conductive strips are incorporated into the non-imprint portions of the garment. The first conductive strips can have a different conductivity than the second conductive strips. The garment portion 1102, 1202 and the window portion 1104 can be arranged such that the inside surface of the window portion 1104 makes contact with the outside surface of the garment portion 1102, 1202. As the garment portion 1102, 1202 stretches, different portions (e.g., the imprints 1106, 1206 and the non-imprint portions) make contact with the window portion 1104. Measurements made by the conductivity sensors depend on the conductivity of the materials in contact with the sensors. Thus, the conductivity measurements can be used to determine the position of the imprints 1106, 1206 and the non-imprint portions in relation to the window portion 1104. In this way, the conductivity measurements can represent the same or similar information described above with respect to the optical sensors.

In some implementations, a grid of conductive strips can be incorporated into the garment portion 1102, 1202 in one direction and a grid of conductivity sensors can be incorporated into the window portion 1104 in a direction perpendicular to the conductive strips. As the garment stretches, the conductive strips in the garment portion 1102, 1202 slide across the conductivity sensors in the window portion 1104. The window portions 1104 does not stretch along with the garment. The measurements from the conductivity sensors can be used to determine a magnitude of stretch experienced by the garment.

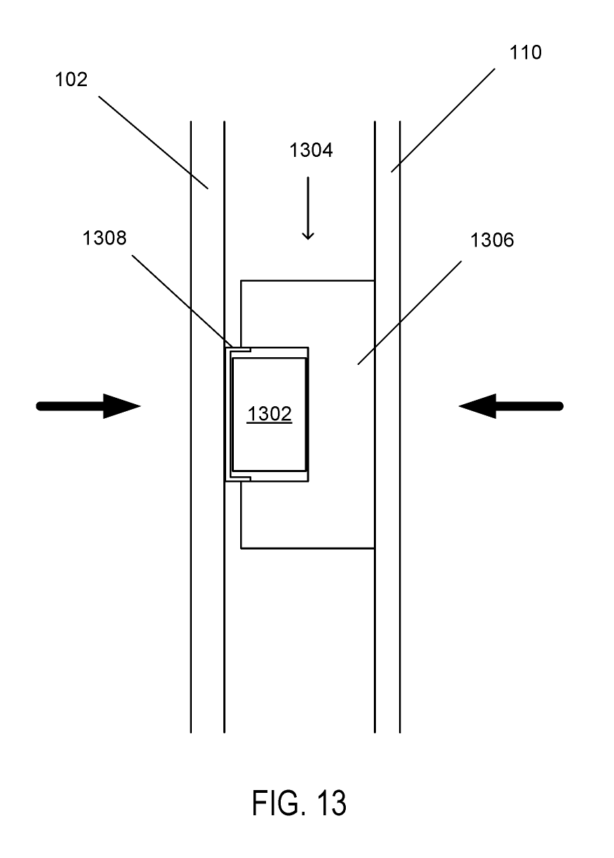

In some implementations, the wearable medical device includes one or more pressure sensors. FIG. 13 shows an example of a pressure sensor 1302 that is incorporated into an electrode 1304 (e.g., a sensing electrode 112 or a treatment electrode 114) of the wearable medical device. The pressure sensor 1302 resides between a first portion 1306 and a second portion 1308 of the electrode 1304. The electrode 1304 is affixed to the garment 110 at the first portion 1306. The electrode 1304 resides between the garment 110 and the patient 102 when the patient 102 is wearing the garment 110 such that the second portion 1308 of the electrode 1304 is in contact with the skin of the patient 1308. The elasticity of the garment 110 causes the electrode 1304 and the pressure sensor 1302 to experience tension. The degree of tension can be measured by the pressure sensor 1302.

Measurements made by the pressure sensor 1302 can be provided to the garment monitoring component 170, and the garment monitoring component 170 can infer a state of the garment 110 based on the measurements. For example, if one or more of the pressure measurements meet or exceed a predetermined threshold, the garment monitoring component 170 may determine that the garment 110 is too tight. In response, the garment monitoring component 170 may cause a message to be provided (e.g., by the touch screen 220 of the controller 120) indicating that the garment 110 should be loosened. If the garment 110 is unable to be loosened, the message may indicate that the garment 110 should be replaced with a larger sized garment. If one or more of the pressure measurements meet or fall below a predetermined threshold, the garment monitoring component 170 may determine that the garment 110 is too loose (e.g., due to excessive continuous or substantially continuous wear). In response, the garment monitoring component 170 may cause a message to be provided indicating that the garment 110 should be tightened and/or laundered and/or replaced with a smaller sized garment. In some implementations, the messages are provided by the speaker 204 of the controller 120.

In some implementations, the pressure sensor 1302 includes a force collector (e.g., such as diaphragm, a piston, a bourdon tube, or bellows) that is configured to measure strain (or, e.g., deflection) due to the force (e.g., pressure or tension) applied over an area of the electrode 1304. In some implementations, the pressure sensor 1302 includes one or more of a capacitive pressure sensor, a capacitive pressure sensor, an electromagnetic pressure sensor, a piezoelectric pressure sensor, an optical pressure sensor, and/or a potentiometric pressure sensor.

In some implementations, pressure sensor 1302 includes a strain gauge (e.g., a piezoresistive strain gauge). The strain gauge can include a strain sensitive metal foil pattern. When pressure is applied to the electrode 1304, the metal foil deforms, causing the overall length of the metal foil pattern to change. The physical change in the metal foil pattern causes the end-to-end resistance of the pattern to change. An output voltage across terminals of the metal foil pattern can correspond to the change of resistance, and therefore is indicative of an amount of strain (e.g., pressure) measured. The output voltage measurement can be provided to the garment monitoring component 170, and the garment monitoring component 170 can determine whether the output voltage meets or exceeds (or, e.g., meets or falls below) a predetermined threshold. The predetermined threshold can correspond to a pressure that is sufficient for keeping the electrode 1304 firmly pressed against the skin of the patient 102 such that the electrode 1304 is held in a substantially fixed position. As described above, confirming that the electrode 1304 is in intimate contact with the patient 110 ensures that strong cardiac signals with minimal noise artifacts are received and/or efficient treatment is delivered.

In some implementations, one or more of the sensing electrodes and/or one or more of the treatment electrodes (e.g., 112 and 114 of FIG. 1) may include a sensor for measuring the conductivity and/or impedance of the interface between the electrode and the patient's skin (e.g., a local surface conductivity and/or impedance measurement of the electrode/skin interface). For example, the conductivity sensor may measure the conductivity and/or impedance between a first point on the surface of the electrode and a second point on the surface of the electrode. When the electrode is firmly pressed against the skin of the patient, the electrode/skin interface typically has a relatively high conductivity (and thus a relatively low impedance, e.g., approximately 110-120 ohms). When the electrode is not firmly pressed against the skin of the patient, the electrode/skin interface typically has a relatively low conductivity (and thus a relatively high impedance, e.g., greater than 140 ohms). Thus, the intimacy of the contact between the electrodes can be determined by measurements from such a conductivity sensor. In some implementations, the conductivity sensor may have a configuration and operate in a fashion similar to that described above with respect to the conductivity meshes 802a-d of FIG. 8.

Measurements from the conductivity sensor(s) may be provided to the garment monitoring component 170 and used to determine the condition and/or state of the garment 110. For example, if the conductivity and/or impedance measured by the conductivity sensor meets or transgresses a threshold (e.g., if a measured impedance exceeds approximately 140 ohms), it can be inferred that the garment 110 has an insufficient elasticity for firmly pressing the electrode against the patient's skin. Thus, the garment 110 may require laundering and/or replacement.

In some implementations, the measurements from the conductivity sensor(s) can be used for falloff detection (e.g., determining whether one or more electrodes has fallen off the patient and/or is about to fall off the patient). In some examples, one of the therapy electrodes 114 may send a signal to another one of the therapy electrodes 114 via the patient's body. The transmitted signal can be compared to the received signal to determine the impedance between the electrodes (e.g., the impedance of the body of the patient). When the measured impedance exceeds a predetermined threshold (e.g., 140 ohms), the medical device may determine that an electrode has fallen off the patient or is about to fall off the patient (e.g., due to the garment 110 having an insufficient elasticity).

In some implementations, the measurements from the conductivity sensor(s) can be used to adaptively adjust the falloff detection threshold in accordance with the condition of the garment 110. For example, if the garment 110 is in like-new condition, a threshold of 120 ohms may be appropriate for falloff detection. However, as the garment 110 gradually loses elasticity either permanently or temporarily due to excessive wear, the impedances measured by the conductivity sensor(s) may generally increase. For example, the garment 110 may have lost some elasticity, but may still be in sufficiently good condition for proper operation of the medical device. Due to the decreased elasticity, the measured impedances may be generally higher (e.g., approximately 140 ohms), yet the electrodes may be sufficiently pressed against the patient's skin. Thus, such measurements may be used to cause the falloff detection threshold to adaptively increase accordingly. In this example, the falloff detection threshold may be increased to 150 ohms, such that measured impedances in excess of 150 ohms indicate that an electrode has fallen off the patient or is about to fall off the patient. In some implementations, the measured impedances may be considered along with measurements from one or more of the garment monitoring sensors 324 for falloff detection purposes.

Adaptive Learning

In some implementations, one or more of the garment monitoring sensors 324 may be used to adjust treatment parameters and/or timings of treatments provided by the medical device. Examples of adjusting treatment parameters of a medical device based on patient, environmental, and/or contextual conditions are described in co-pending U.S. Provisional Application No. 62/235,165, filed Sep. 30, 2015, entitled “MEDICAL DEVICE OPERATIONAL MODES”, included herein as Appendix A, and the contents of which are incorporated herein in their entirety.

In some implementations, measurements from the temperature sensor 326 and/or the humidity sensor 328 can be used to modify the parameters for determining whether the patient is experiencing a cardiac event. When determining whether the patient is experiencing a cardiac event, it may be important to distinguish between actual cardiac signals and noise components, which may sometimes look similar to cardiac signals. Measurements from the temperature sensor 326 and/or the humidity sensor 328, as well as measurements from other garment monitoring sensors 324 or additional sensors incorporated into the medical device, can be used to ascertain patient conditions, environmental condition, and/or contextual conditions. Such conditions may typically be associated with increased noise in the cardiac signal. For example, cold and/or dry weather may typically cause increased noise in sensed cardiac signals (e.g., due to less intimacy leading to, for instance, higher impedance between a sensing electrode and the patient's skin). Measurements from the temperature sensor 326 and/or the humidity sensor 328 can be used to detect cold and/or dry conditions, and noise detection algorithms can be adjusted accordingly. For example, a threshold associated with noise detection can be increased to reduce false positives indicating an unacceptable amount of noise. In some implementations, an amount of time before which a treatment is applied (e.g., if the patient does not interact with the response buttons 210 of FIGS. 2A-2B) may be extended to provide the patient with a longer opportunity to stop the treatment from being applied.

In some implementations, measurements from the temperature sensor 326 and/or the humidity sensor 328 can be used to determine a patient condition and/or a contextual condition. Such measurements may be used to determine that the patient is in an environment with water (e.g., the patient is in the shower), and parameters for determining whether the patient is experiencing a cardiac event may be adjusted in response. For example, a patient who is showering may lift or cause excessive movement of the electrodes, thereby resulting in excess noise artifacts. Thresholds associated with noise detection can be increased to reduce false positives indicating an unacceptable amount of noise.

Notifications

The garment monitoring component 170 is configured to cause messages (e.g., including alerts, notifications, alarms, sounds, voice prompts, etc.) to be provided related to the identity of the garment, the laundering of the garment, and/or the state (e.g., condition) of the garment. The messages can be provided by a user interface of the medical device controller 120.

In some implementations, the controller 120 can provide messages related to the identity of the garment. For example, briefly referring back to FIG. 1, when the garment identification component (e.g., the RFID tag 150) of the garment 110 provides information to the RFID reader 160, the controller 120 can present one or more messages related to the provided information to indicate that the garment 110 is being associated with the controller 120. The message may include identification information that corresponds to the garment 110, such as the identification information described above. The message can be presented on the touch screen (220 of FIGS. 2A-2B) of the controller 120.

In some implementations, a message is presented by the controller 120 when a garment 110 is associated and/or disassociated with the controller 120. In some implementations, a message is presented by the controller 120 when a garment 110 has been associated (e.g., continuously or substantially continuously) with the controller 120 for an amount of time that meets or exceeds a predetermined threshold, thereby notifying the patient that the garment 110 should be removed or replaced (e.g., with a second garment). In some implementations, the message may suggest that the patient launder the garment 110 in an attempt to restore the garment 110 to like-new condition.

In some implementations, the controller 120 can provide messages related to the laundering of the garment. For example, when the garment monitoring component 170 determines that a laundering event has occurred (e.g., based on measurements by the temperature sensor 326 and/or the humidity sensor 328 among others, as described in detail above), the controller 120 can provide a message indicating such (e.g., “Garment Laundering Detected”). Similarly, if a laundering event has not been detected by the garment laundering component 170 for a particular amount of time, the controller 120 can provide a message indicating such (e.g., “Garment Has Not Been Laundered for X Days”), thereby reminding the patient that garment laundering is overdue. In some implementations, the message includes a recommendation to remove, replace, and/or launder the garment.

In some implementations, when a laundering event is detected, the controller 120 is configured to provide a message that includes a prompt that the patient can interact with to confirm or refute that a laundering event has occurred. For example, under some circumstances, the garment monitoring component 170 may identify a laundering event, but the identification may be erroneous. The controller 120 can provide a message “Garment Laundering Detected—Confirm?” that includes a prompt that provides “Yes” and “No” input elements. The patient can interact with the “No” input element to inform the controller 120 that that garment is not currently being laundered and/or has already been laundered. In some implementations, refuting an erroneous garment laundering detection event can cause the garment monitoring component 170 to adjust its methodology for detecting laundering events (e.g., by adjusting predetermined thresholds for temperature and/or humidity values).

In some implementations, the controller 120 can provide messages related to the state (e.g., condition) of the garment. The message can include information related to one or more measurements made by any combination of the various garment monitoring sensors (324 of FIG. 3) described above and hereafter. For example, the message can include information related to a measured temperature, humidity, pH, strain (e.g., inductance and/or capacitance), and/or tear (e.g., current and/or resistance), among others. In some implementations, some or all of the information included in such messages can also be provided for technical support and/or troubleshooting purposes (e.g., remote troubleshooting). In some examples, the information may be provided to a server associated with the manufacturer of the controller 120 and/or the garment. Such information may be referenced in the future, such as when a patient calls technical support to report and seek help with an issue. In some implementations, receipt of this information may trigger one or more actions. For example, in some implementations, information indicating that the condition of the garment may be unsatisfactory, or information indicating that the condition of the garment may soon be unsatisfactory, may trigger an automatic reordering of a new garment. In this way, a replacement garment can be automatically provided with minimal work and/or involvement on the part of the patient.

In some implementations, the controller 120 is configured to provide a message when the garment monitoring component 170 determines that the garment is in an unsatisfactory state (e.g., based on measurements made by the garment monitoring sensors 324). In some implementations, the message includes information related to a reason why the garment monitoring component 170 determined that the garment is in an unsatisfactory condition. For example, the message may state that that the garment is in an unsatisfactory condition because the strain sensor 330 measured a stretch value (e.g., based on a measured inductance and/or capacitance) that exceeded a predetermined threshold, thereby indicating that the electrodes might not be in firm contact against the patient's body. The message may state that the garment is in an unsatisfactory condition because the tear sensor 332 measured a current and/or resistance value that exceeded a predetermined threshold, thereby indicating that a tear may be present in the garment. In some implementations, the message includes a recommendation for correcting the identified issue. For example, the message may suggest that the patient tighten and/or launder the garment if the elasticity of the garment is determined to be insufficient. The message may suggest that the garment be replaced if it is determined that the garment is developing or has developed a tear. In some implementations, the recommendation is based on a history of measurements made by the garment monitoring sensors 324 (e.g., measurements stored in the event log 1000 of FIG. 10). For example, if the historical measurements indicate that the garment no longer sufficiently restores to its baseline elasticity when it is laundered, the message can recommend that the garment be discarded and replaced with a new garment.

In some implementations, the controller 120 is configured to provide messages (e.g., audio messages) through the speaker 204 instead of or in addition to messages provided by the touch screen 220. The audio messages can include the same or similar content as that described above with respect to text-based messages. In some implementations, a haptic alert can be provided by the wearable medical device 100 (e.g., by a vibration motor incorporated into the connection pod 130) in conjunction with the message to notify the patient that a message has been provided.

As briefly mentioned above, the wearable medical device 100 is configured to transmit information to a server (e.g., the remote server 322 of FIG. 3) in communication with a network (e.g., via the network interface 306). The network interface 306 can facilitate the communication of information between the controller 120 and one or more other devices or entities over a communications network.

In some implementations, the controller 120 can transmit information related to the identity of the garment. For example, the controller 120 can transmit identification information of all garments that have been associated with the controller 120, particular times at which each garment became associated with the controller 120, and/or particular times at which each garment became disassociated with the controller 120, among others. In some implementations, the controller 120 can transmit data indicative of whether a garment has been removed on at least a predetermined interval.

In some implementations, the controller 120 can transmit information related to the laundering of the garment. For example, the controller 120 can transmit information related to laundering events that are detected, including information related to one or more measurements (e.g., from the garment monitoring sensors 324) that lead to the determination. The controller 120 can transmit information indicating that a laundering event has not been detected for a particular amount of time. A recipient of the transmitted information can contact the patient and suggest that the garment be laundered.

In some implementations, the controller 120 can transmit information related to the state of the garment. For example, the controller 120 can transmit information indicating that the garment is in an unsatisfactory state (e.g., based on measurements made by the garment monitoring sensors 324). In some implementations, the controller 120 can transmit measurements made by the various sensors (e.g., the garment monitoring sensors 324) described herein. The transmitted information may be compiled into an event log (e.g., the event log 1000 of FIG. 10). The information can be used to correlate the state of the garment and/or laundering events with the various sensor measurements. For example, using measurements made by the temperature sensor 326, the humidity sensor 328, and the strain sensor 330, preferred laundering conditions that effectively restore the garment to its baseline elasticity can be identified. For example, using measurements made by the temperature sensor 326, the humidity sensor 328, and the tear sensor 332, environmental conditions that result in degraded garment quality (e.g., as indicated by the detection of a tear) can be identified.

While certain implementations have been described, other implementations are possible.

While the components of the communication interface (e.g., including the NFC elements, the RFID elements, etc.) have been described as being incorporated into the garment and/or the connection pod, such components may be located elsewhere on the medical device. For example, in some implementations, the RFID reader may be incorporated as part of the medical device controller. In some implementations, the RFID reader may be incorporated as part of a separate device (e.g., as part of a mobile computing device such as a smartphone or tablet). The RFID reader and/or the mobile computing device may be configured to associate with (e.g., pair with) the controller. In this way, the RFID reader of the mobile computing device can be used to associate the RFID tag corresponds to a particular garment with the controller.

While the garment monitoring component has been described as being disposed as part of the garment, in some implementations, the garment monitoring component can be incorporated into the controller. In some implementations, the garment monitoring sensors can be configured to store collected data and provide the data to the garment monitoring component at a later time. For example, the garment and the garment monitoring sensors can be removed from the rest of the wearable medical device (e.g., unattached from the controller) for laundering, and the garment monitoring sensors can provide the collected data to the garment monitoring component when the garment monitoring sensors are reattached to the controller (e.g., via the connection pod).

In some implementations, the information received by the strain sensor (e.g., including the inductive element and/or the capacitive element) can be used to measure a respiration rate of the patient. When the patient inhales, the strain sensor provides a measurement indicative of an increased stretch experienced by the garment, and when the patient exhales, the strain sensor provides a measurement indicative of a decreased stretch experienced by the garment. The controller can determine the respiration rate of the subject based on the timings of stretch value increases and decreases. For example, if the stretch values are increasing and decreasing according to a rhythm that would typically correspond to a respiration rate, the controller can infer that the stretch value increases and decreases are due to the patient's breathing and determine the patient's respiration rate accordingly.

In some implementations, the patient's respiration rate can be compared to other information, such as the patient's ECG, to infer a state of the garment. For example, the patient' s respiration rate can be determined based on i) the patient's ECG signal, and ii) the timings of increasing/decreasing stretch values derived from the strain sensor. The respiration rates determined by each technique can be compared to each other, and if each of the respiration rates substantially match, the controller can infer that the garment has a proper fit for the patient. On the other hand, if the respiration rates do not substantially match, the controller can infer that the garment does not have a proper fit for the patient. For example, if the garment has become too loose fitting (e.g., due to excessive continuous wear), the respiration rate as determined by the increasing/decreasing stretch values may be inaccurate.

In some implementations, the confidence of a determination made by the controller (e.g., a determination that the patient is experiencing a health-related event) may be based at least in part on the state of the garment. For example, if the controller identifies that the respiration rate determined based on the measurements from the strain sensor is inaccurate (thereby indicating that the state of the garment may be inadequate), the controller may have reduced confidence in a determination that the patient has stopped breathing because the determination may be based on the garment having too loose of a fit. If the controller identifies that the respiration rate determined based on the measurements from the strain sensor is accurate (thereby indicating that the state of the garment is sufficient), the controller may have increased confidence in a determination that the patient has stopped breathing because the determination is likely not based on the garment not having a proper fit. In some implementations, the confidence that the controller has in a determination may affect one or more operating parameters of the medical device. For example, if the medical device is relatively confident in a determination, a shorter warning may be provided to the patient before a treatment is applied.

In some implementations, the wearable medical device can include fewer or additional sensors (e.g., additional garment monitoring sensors).

In some implementations, the wearable medical device includes an odor sensor (e.g., an odor meter or an electric nose). The odor sensor can be incorporated into the garment or into some other portion of the wearable medical device (e.g., the connection pod). The odor sensor is configured to detect an intensity of the patient's body odor and provide measurements to the garment monitoring component. If the intensity of the patient's body odor meets or exceeds a predetermined threshold, the garment monitoring component can infer that the garment has not been changed and/or laundered. The garment monitoring component can then cause a message to be provided indicating that the garment should be changed and/or laundered.

In some implementations, the odor sensor includes a metal-oxide-semiconductor (MOSFET) device that includes a transistor. Molecules (e.g., molecules that produce odor) in proximity to the sensor have a positive or negative charge. The particular charge of the molecules has a direct effect on the electric field inside the MOSFET. Thus, as additional charged molecules are introduced to the transistor, the signal produced by the MOSFET changes. The signal can be provided to pattern recognition software to determine characteristics of the charged molecules, including the intensity of odors produced by the charged molecules. In some implementations, the odor sensor also or instead includes one or more of a conducting organic polymer sensor, a polymer composite sensor, a quartz crystal microbalance sensor, and/or a surface acoustic wave (SAW) sensor, among others.

In some implementations, the wearable medical device includes a color detector (e.g., a dirt detector). The color detector is configured to make visual measurements related to a color of the garment to determine whether the garment is dirty. For example, the garment may be white when it is clean, but after a period of continuous use, the garment may turn gray or brown as it becomes dirty. The cleanliness of the garment can be used to infer how long the garment has been continuously (or substantially continuously) worn, and thus can be an indicator of when the garment should be laundered or replaced. The garment monitoring component can then cause a message to be provided indicating that the garment should be changed and/or laundered.

In some implementations, the color sensor can include a light source and an optical sensor. The light source can emit a white light onto the garment, and the optical sensor can measure the light that is reflected off of the garment. The optical sensor can determine the color of the garment based on characteristics of the measured reflected light. If the measurements indicate that the garment includes brown or gray colors in an amount and/or intensity that exceeds a predetermined threshold, the garment monitoring component can infer that the garment has been continuously (or substantially continuously) worn for too long and thus should be laundered or replaced. The optical sensor can be configured to measure colors other than brown or gray that are indicative of dirt being present. In some implementations, the color sensor is incorporated into the connection pod of the wearable medical device.

In some implementations, the wearable medical device includes a skin detector that is configured to identify the patient's skin (e.g., to determine that the garment is currently being worn). The skin detector can be incorporated into the garment. In some implementations, the skin detector includes one or both of an inductive sensor and a capacitive sensor. The patient's skin has electrical properties that can be detected by the inductive sensor and/or the capacitive sensor. When the inductive sensor and/or the capacitive sensor are in contact with the skin, electrical properties measured by the sensors is affected. Using these measurements, the skin detector can determine whether it is in contact with the patient's skin. The garment monitoring component can use this information to determine a length of time (and, e.g., the particular times) that the garment is being worn, as well as particular times at which the garment is put on and/or removed. If the garment monitoring component determines that the garment has been continuously (or substantially continuously) worn for an amount of time that meets or exceeds a predetermined threshold, the garment monitoring component can cause a message to be provided indicating that the garment should be changed and/or laundered.

In some implementations, the components described with reference to FIG. 3 (e.g., the cardiac event detector 320) can be implemented using hardware or a combination of hardware and software. For example, in some implementations, the cardiac event detector 320 is implemented as a software component that is stored within the data storage and executed by the processor.

In some implementations, the medical devices described herein can include one or more additional sensors and corresponding sensor interface components. Examples of additional sensors can include a body temperature sensor, a respiration sensor, an environmental sensor (e.g., an atmospheric thermometer, an airflow sensor, a video sensor, an audio sensor, a locational sensor, a hygrometer, etc.), or a motion sensors (e.g., an accelerometer, a gyroscope, etc.). In some implementations, the additional sensors are wirelessly coupled to the medical device controller.

In some implementations, the medical devices can include one or more additional therapy delivery components (e.g., in addition to therapy electrodes) and corresponding therapy delivery interface components. Examples of additional therapy delivery components can include a capacitor, a pacing electrode, and/or a mechanical chest compression device. In some implementations, the additional therapy delivery components are wirelessly coupled to the medical device controller.

While the medical device controller has been described as including a processor, in some implementations, the processor is included in another portion of the medical device (e.g., outside of the medical device controller). In some implementations, the medical device includes multiple processors, any of which may be located inside or outside of the medical device controller. In some implementations, a processor is included in an external pod (e.g., the connection pod 130 of FIG. 1.)

While the medical devices described herein have been described and shown as including a particular number of electrodes positioned at particular locations on the patient, the number and/or positions of the electrodes can vary to best suit the particular application.

In some implementations, the medical devices described herein are configured to communicate with another device (e.g., a smartphone, a personal digital assistant, a tablet, etc.) over a network. The communication network may be wired or wireless.

### Example Infrastructure

Software running on the medical device controller (e.g., controller 120 of FIGS. 1-4) can be realized by instructions that upon execution cause one or more processing devices to carry out the processes and functions described above, for example, determining how long a garment has been worn, determining whether a garment has been removed or replaced according to a predetermined schedule, determining whether a garment has been removed on at least a predetermined interval, determining whether a garment has been in a laundering environment, determining whether a garment has been laundered according to a predetermined schedule, determining information related to a state of the garment, determining whether a garment is in an unsatisfactory condition, determining whether a garment is being cared for according to predetermined criteria, and determining whether measurements made by one or more garment monitoring components meet or exceed respective predetermined thresholds, among others. Such instructions can include, for example, interpreted instructions such as script instructions, or executable code, or other instructions stored in a computer readable medium.

A server (e.g., the remote server 322 as shown in FIG. 3) can be distributively implemented over a network, such as a server farm, or a set of widely distributed servers or can be implemented in a single virtual device that includes multiple distributed devices that operate in coordination with one another. For example, one of the devices can control the other devices, or the devices may operate under a set of coordinated rules or protocols, or the devices may be coordinated in another fashion. The coordinated operation of the multiple distributed devices presents the appearance of operating as a single device.

In some examples, the components of the controller 120 as shown in FIGS. 2-4 may be contained within a single integrated circuit package. A system of this kind, in which both a processor (e.g., the processor 318) and one or more other components (e.g., the cardiac event detector 320) are contained within a single integrated circuit package and/or fabricated as a single integrated circuit, is sometimes called a microcontroller. In some implementations, the integrated circuit package includes pins that correspond to input/output ports (e.g., that can be used to communicate signals to and from one or more of the input/output interface devices).

Although an example processing system has been described above, implementations of the subject matter and the functional operations described above can be implemented in other types of digital electronic circuitry, or in computer software, firmware, or hardware, including the structures disclosed in this specification and their structural equivalents, or in combinations of one or more of them. Implementations of the subject matter described in this specification, such as storing, maintaining, and displaying artifacts can be implemented as one or more computer program products, i.e., one or more modules of computer program instructions encoded on a tangible program carrier, for example a computer-readable medium (e.g., the data storage 304), for execution by, or to control the operation of, a processing system. The computer readable medium can be a machine readable storage device, a machine readable storage substrate, a memory device, or a combination of one or more of them.

The term “system” may encompass all apparatus, devices, and machines for processing data, including by way of example a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them. In some implementations, operating systems can include a Windows based operating system, OSX, or other operating systems. For instance, in some examples, the processor may be configured to execute a real-time operating system (RTOS), such as RTLinux, or a non-real time operating system, such as B SD or GNU/Linux.

A computer program (also known as a program, software, software application, script, executable logic, or code) can be written in any form of programming language, including compiled or interpreted languages, or declarative or procedural languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

Computer readable media suitable for storing computer program instructions and data include all forms of non-volatile or volatile memory, media and memory devices, including by way of example semiconductor memory devices, e.g., EPROM, EEPROM, and flash memory devices; magnetic disks, e.g., internal hard disks or removable disks or magnetic tapes; magneto optical disks; and CD-ROM, DVD-ROM, and Blu-Ray disks. The processor and the memory can be supplemented by, or incorporated in, special purpose logic circuitry. Sometimes a server (e.g., the remote server 322 as shown in FIG. 3) is a general purpose computer, and sometimes it is a custom-tailored special purpose electronic device, and sometimes it is a combination of these things. Implementations can include a back end component, e.g., a data server, or a middleware component, e.g., an application server, or a front end component, e.g., a client computer having a graphical user interface or a Web browser through which a user can interact with an implementation of the subject matter described is this specification, or any combination of one or more such back end, middleware, or front end components. The components of the system can be interconnected by any form or medium of digital data communication, e.g., a communication network such as the connection between the remote server 322 and the network interface 306 shown in FIG. 3. Examples of communication networks include a local area network (“LAN”) and a wide area network (“WAN”), e.g., the Internet.

Having described several aspects of at least one example of this disclosure, the examples of the methods and apparatuses discussed herein are not limited in application to the details of construction and the arrangement of components set forth in this description or illustrated in the accompanying drawings. The methods and apparatuses are capable of implementation in other examples and of being practiced or of being carried out in various ways. Examples of specific implementations are provided herein for illustrative purposes only and are not intended to be limiting. In particular, acts, elements and features discussed in connection with any one or more examples are not intended to be excluded from a similar role in any other examples. Accordingly, the foregoing description and drawings are by way of example only

Also, the phraseology and terminology used herein is for the purpose of description and should not be regarded as limiting. Any references to examples or elements or acts of the systems and methods herein referred to in the singular may also embrace examples including a plurality of these elements, and any references in plural to any example or element or act herein may also embrace examples including only a single element. References in the singular or plural form are not intended to limit the presently disclosed systems or methods, their components, acts, or elements. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms.

What is claimed is:

1. An apparatus comprising:
a first garment configured to be worn about a torso of a patient; and
a first garment identification component disposed as a part of the first garment, the first garment identification component configured to operably couple with a medical device controller, wherein the medical device controller is configured to identify the first garment based on one or more values provided by the first garment identification component.

2. The apparatus of claim 1, wherein the medical device controller is configured to associate with the first garment after identifying the first garment.

3. The apparatus of claim 1, wherein the apparatus comprises a wearable medical device.

4. The apparatus of claim 1, wherein the first garment identification component comprises at least one of a near field communication (NFC) element and a machine-readable data element.

5. The apparatus of claim 4, wherein the NFC element comprises a radio-frequency identification (RFID) element and the machine-readable data element comprises a barcode.

6. The apparatus of claim 2, comprising the medical device controller, wherein the medical device controller is configured to determine an amount of time that the first garment has been worn by the patient.

7. The apparatus of claim 6, wherein the medical device controller is configured to determine an amount of time that the first garment has been worn by the patient by determining an amount of time that the first garment has been associated with the medical device controller without the medical device controller being associated with another garment.

8. The apparatus of claim 7, wherein the medical device controller is configured to determine that the first garment is to be removed or replaced based on the amount of time that the first garment has been associated with the medical device controller without the medical device controller being associated with another garment.

9. The apparatus of claim 8, wherein the medical device controller is configured to determine that the first garment is to be removed or replaced if the amount of time meets a predetermined threshold amount of time.

10. The apparatus of claim 6, wherein the medical device controller is configured to operably couple with a second garment identification component disposed as a part of a second garment configured to be worn about the torso of the patient, wherein the medical device controller is configured to identify the second garment based on one or more values provided by the second garment identification component.

11. The apparatus of claim 10, wherein the medical device controller is configured to disassociate the medical device controller from the first garment and associate the medical device controller with the second garment upon the medical device controller identifying the second garment.

12. The apparatus of claim 10, wherein the medical device controller is configured to, upon identifying the second garment, provide a prompt to a user to enable the user to cause the medical device controller to disassociate the medical device controller from the first garment and associate the medical device controller with the second garment.

13. A wearable medical device comprising:
a controller;
a memory storing a first value identifying a first garment configured to couple with the controller;
a communication interface; and
one or more processors configured for
receiving, from the communication interface, a second value identifying a second garment configured to couple with the controller,
comparing the first value and the second value, and
based on the comparison of the first value and the second value, storing data indicative of whether the first garment has been removed on at least a predetermined interval.

14. The wearable medical device of claim 13, wherein one or both of the first value identifying the first garment and the second value identifying the second garment is unique to the respective garment and comprises data that corresponds to an identity of the respective garment, the data corresponding to one or more of a serial number of the respective garment, a model number of the respective garment, and a size of the respective garment.

15. The wearable medical device of claim 13, wherein the data indicative of whether the first garment has been removed on at least a predetermined interval comprises data indicative of whether the first garment has been replaced with the second garment on at least a predetermined interval.

16. The wearable medical device of claim 13, comprising a user interface configured to provide one or more messages based at least in part on the data indicative of whether the first garment has been removed on at least a predetermined interval.

17. The wearable medical device of claim 13, wherein the one or more processors are configured for calculating the data indicative of whether the first garment has been removed on at least a predetermined interval, based at least on the second value.

18. The wearable medical device of claim 17, comprising a network communication interface configured to transmit, to a server in communication with a network, the data indicative of whether the first garment has been removed on at least a predetermined interval.

19. An apparatus comprising:
a garment configured to be worn about a torso of a patient;
a garment monitoring component disposed as a part of the garment and configured to determine whether the garment has been laundered according to a predetermined schedule; and
a communication device operably connected to the garment monitoring component and configured to exchange information related to the laundering of the garment with a medical device controller.

20. The apparatus of claim 19, wherein the garment monitoring component comprises at least one sensor that measures at least one of temperature, humidity, pH, and fabric stretch.

21. The apparatus of claim 19, wherein the garment monitoring component is configured to determine whether the garment has been laundered according to a predetermined schedule based at least in part on two or more measured values.

22. The apparatus of claim 21, wherein the two or more measured values are temperature values measured by a temperature sensor, and the determination of whether the garment has been laundered according to the predetermined schedule is based on whether any of the temperature values meets a predetermined threshold.

23. The apparatus of claim 22, wherein a temperature value that meets the predetermined threshold is indicative of the garment having been in a laundering environment.

24. The apparatus of claim 21, wherein the two or more measured values are humidity values measured by a humidity sensor, and the determination of whether the garment has been laundered according to the predetermined schedule is based on whether any of the humidity values meets a predetermined threshold.

25. The apparatus of claim 24, wherein a humidity value that meets the predetermined threshold is indicative of the garment having been in a laundering environment.

26. The apparatus of claim 21, wherein the two or more measured values are pH values measured by a pH sensor, and the determination of whether the garment has been laundered according to the predetermined schedule is based on whether any of the pH values meets a predetermined threshold.

27. The apparatus of claim 26, wherein a pH value that meets the predetermined threshold is indicative of the garment having been in a laundering environment.

28. The apparatus of claim 21, wherein the two or more measured values are stretch values measured by a strain sensor, and the determination of whether the garment has been laundered according to the predetermined schedule is based on whether any of the stretch values meets a predetermined threshold.

29. The apparatus of claim 28, wherein a stretch value that meets the predetermined threshold is indicative of the garment having not been in a laundering environment.

30. The apparatus of claim 28, wherein a difference between a first stretch value and a second stretch value that meets the predetermined threshold is indicative of the garment having been in a laundering environment.

31. The apparatus of claim 28, wherein the strain sensor comprises a coil embedded in fabric of the garment.

32. The apparatus of claim 21, wherein the two or more measured values include one or both of an inductance value and a capacitance value indicative of stretch experienced by the garment.

33. The apparatus of claim 19, comprising the medical device controller, wherein the medical device controller comprises a user interface configured to provide one or more messages based at least in part on the information related to the laundering of the garment.

34. The apparatus of claim 19, wherein the communication device is configured to transmit, to a server in communication with a network, the information related to the laundering of the garment.

35. The apparatus of claim 19, wherein the information related to the laundering of the garment includes one or more of a measured temperature value, humidity value, pH value, and stretch value, and one or more laundering events experienced by the garment are identified based on the information related to the laundering of the garment.

36. The apparatus of claim 19, wherein the communication device operates according to an NFC protocol.

37. An apparatus comprising:
a garment configured to be worn about a torso of a patient;
a garment monitoring component disposed as a part of the garment and configured to monitor a state of the garment; and
a communication device operably connected to the garment monitoring component and configured to exchange information related to the state of the garment with a medical device controller.

38. The apparatus of claim 37, wherein the apparatus comprises a wearable medical device.

39. The apparatus of claim 37, wherein monitoring the state of the garment includes determining whether the garment is being cared for according to predetermined criteria.

40. The apparatus of claim 37, comprising at least one sensing electrode for sensing a physiological condition of the patient.

41. The apparatus of claim 37, comprising at least two cardiac sensing electrodes for sensing a cardiac condition of the patient.

42. The apparatus of claim 37, comprising at least two therapy electrodes for delivering a therapy to the patient.

43. The apparatus of claim 37, comprising:
at least two sensing electrodes for sensing a cardiac arrhythmia condition of the patient; and
at least two therapy electrodes for delivering at least one therapeutic pulse to the patient in response to the sensed cardiac arrhythmia condition.

44. The apparatus of claim 37, comprising a coil embedded in fabric of the garment, the coil configured to provide, to the garment monitoring component, information related to a magnitude of stretch experienced by the garment, wherein the garment monitoring component is configured to monitor the state of the garment based at least in part on the information related to the magnitude of stretch.

45. The apparatus of claim 37, comprising a tear sensor embedded in fabric of the garment, the tear sensor configured to provide, to the garment monitoring component, information related to a tear in the garment, wherein the garment monitoring component is configured to monitor the state of the garment based at least in part on the information related to the tear in the garment.

46. The apparatus of claim 45, wherein the tear sensor comprises a conductive mesh, and the information related to the tear in the garment is based at least in part on a magnitude of current running through the conductive mesh when a predetermined voltage is applied across the conductive mesh.

47. The apparatus of claim 37, wherein the garment monitoring component is configured to determine whether the garment is in an unsatisfactory condition based at least in part on one or more measured values related to a magnitude of stretch experienced by the garment, a magnitude of current running through a conductive mesh of the garment, or both.

48. The apparatus of claim 37, comprising a slotted window through which a portion of the garment is visible, wherein the visible portion of the garment is indicative of a magnitude of stretch experienced by the garment.

49. The apparatus of claim 48, comprising an optical sensor configured to:
capture an image of the portion of the garment visible through the slotted window; and
provide, to the garment monitoring component, information related to the image,
wherein the information related to the image includes one or more values that represent a color composition of the image.

50. The apparatus of claim 37, comprising the medical device controller, wherein the medical device controller comprises a user interface configured to provide one or more messages based at least in part on the state of the garment.

51. The apparatus of claim 37, wherein the communication device is configured to transmit, to a server in communication with a network, information related to the state of the garment.

52. The apparatus of claim 37, wherein the communication device operates according to an NFC protocol.
